(19) United States
(12) Patent Application Publication
(10) Pub. No.: US 2013/0190314 A1
(43) Pub. Date: Jul. 25, 2013

(54) DERIVATIVES OF 6-CYCLOAMINO-2-THIENYL-3-(PYRIDIN-4-YL)IMIDAZO[1,2-b]-PYRIDAZINE AND 6-CYCLOAMINO-2-FURANYL-3-(PYRIDIN-4-YL)IMIDAZO[1,2-b]-PYRIDAZINE, PREPARATION AND THERAPEUTIC USE THEREOF

(71) Applicants: Yulin Chiang, Bridgewater, NJ (US); Cecile Enguehard-Gueiffier, Tours (FR); Pascal George, Paris (FR); Alaim Gueiffier, Tours (FR); Frederic Puech, Paris (FR); Mireille Sevrin, Paris (FR); Qiuxia Zhao, Bridgewater, NJ (US)

(72) Inventors: Yulin Chiang, Bridgewater, NJ (US); Cecile Enguehard-Gueiffier, Tours (FR); Pascal George, Paris (FR); Alaim Gueiffier, Tours (FR); Frederic Puech, Paris (FR); Mireille Sevrin, Paris (FR); Qiuxia Zhao, Bridgewater, NJ (US)

(21) Appl. No.: 13/141,006

(22) PCT Filed: Dec. 17, 2009
(86) PCT No.: PCT/FR2009/052592

§ 371 Date: Sep. 9, 2011

Related U.S. Application Data
(60) Provisional application No. 61/139,654, filed on Dec. 22, 2008.

(30) Foreign Application Priority Data
Dec. 19, 2008 (FR) 0807260

(51) Int. Cl. C07D 487/04

(52) U.S. Cl. CPC C07D 487/04
USPC 514/248; 544/236; 544/230

(57) ABSTRACT

The invention relates to derivatives of 6-cycloamino-2-thienyl-3-(pyridin-4-yl)imidazo[1,2-b]-pyridazine and 6-cycloamino-2-furanyl-3-(pyridin-4-yl)imidazo[1,2-b]-pyridazine with general formula (I). The invention also relates to a method for the preparation and therapeutic application thereof, in the treatment or prevention of illnesses involving casein kinase 1 epsilon and/or casein kinase 1 delta.

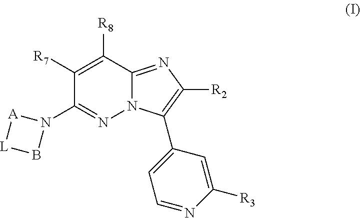

The present invention relates to derivative of 6-cycloamino-2-thienyl-3-(pyridin-4-yl)-imidazo[1,2-b]pyridazine and of 6-cycloamino-2-furanyl-3-(pyridin-4-yl)imidazo-[1,2-b]pyridazine, to the preparation thereof and to the therapeutic use thereof, in the treatment or prevention of diseases involving casein kinase 1 epsilon and/or casein kinase 1 delta.

The subject of the present invention is the compounds corresponding to general formula (I)

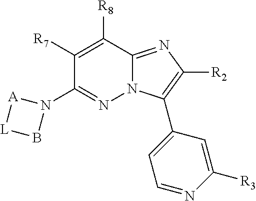

in which:
R₂ is a thienyl group or a furanyl group, optionally substituted with one or more substituents chosen from halogen atoms and C₁₋₆-alkyl groups;
R₃ is a hydrogen atom or a C₁₋₃-alkyl, —NR₄R₅, or C₁₋₄-alkyloxy group;
A is a C₁₋₇-alkylene group optionally substituted with one or two Rₐ groups;
B is a C₁₋₇-alkylene group optionally substituted with an R_{b} group;
L is either a nitrogen atom optionally substituted with an R_{c} or R_{d} group, or a carbon atom substituted with an Rₑ₁ group and an R_{d} group or two Rₑ₂ groups;

the carbon atoms of A and of B being optionally substituted with one or more R_{f} groups, which may be identical to or different from one another;

Rₐ, R_{b} and R_{c} are defined such that:
two Rₐ groups can together form a C₁₋₆-alkylene group;
Rₐ and R_{b} can together form a bond or a C₁₋₆-alkylene group;
Rₐ and R_{c} can together form a bond or a C₁₋₆-alkylene group;
R_{b} and R_{c} can together form a bond or a C₁₋₆-alkylene group;
R_{d} is a group chosen from a hydrogen atom and C₁₋₆-alkyl, C₃₋₇-cycloalkyl, C₃₋₇-cycloalkyl-C₁₋₆-alkyl, C₁₋₆-alkyloxy-C₁₋₆-alkyl, C₁₋₆-fluoroalkyl, benzyl and hydroxy-C₁₋₆-alkyl groups;
Rₑ₁ is an —NR₄R₅ group or a cyclic monoamine optionally comprising an oxygen atom, the cyclic monoamine being optionally substituted with one or more substituents chosen from a fluorine atom and C₁₋₆-alkyl, C₁₋₆-alkyloxy and hydroxyl groups;
Two Rₑ₂ form, with the carbon atom which bears them, a cyclic monoamine optionally comprising an oxygen atom, this cyclic monoamine being optionally substituted with one or more R_{f} groups, which may be identical to or different from one another;
R_{f} is a C₁₋₆-alkyl, C₃₋₇-cycloalkyl, C₁₋₆-alkyloxy-C₁₋₆-alkyl, hydroxy-C₁₋₆-alkyl, C₁₋₆-fluoroalkyl or phenyl group;
R₄ and R₅ are, independently of one another, a hydrogen atom or a C₁₋₄-alkyl, C₃₋₇-cycloalkyl or C₃₋₇-cycloalkyl-C₁₋₆-alkyl group;
   R₇ and R₅ are, independently of one another, a hydrogen atom or a C₁₋₆-alkyl group.

The compounds of formula (I) may comprise one or more asymmetrical carbon atoms. They may therefore exist in the form of enantiomers or of diastereoisomers. These enantiomers and diastereoisomers, and also mixtures thereof, including racemic mixtures, form part of the invention.

The compounds of formula (I) may exist in the form of bases or of addition salts with acids. Such addition salts form part of the invention. These salts are advantageously prepared with pharmaceutically acceptable acids, but the salts of other acids that are useful, for example, for purifying or isolating the compounds of formula (I) also form part of the invention.

The compounds of formula (I) may also exist in the form of hydrates or of solvates, i.e. in the form of associations or combinations with one or more molecules of water or with a solvent. Such hydrates and solvates also form part of the invention.

In the context of the invention:
the term “C_{t-z}”, where t and z may have values from 1 to 7, is intended to mean a carbon-based chain that may contain from t to z carbon atoms, for example the term “C₁₋₇” is intended to mean a carbon-based chain that may contain from 1 to 7 carbon atoms;
the term “alkyl” is intended to mean a linear or branched, saturated aliphatic group; for example, a C₁₋₆-alkyl group is a linear or branched carbon-based chain of 1 to 6 carbon atoms, for example a methyl, ethyl, propyl, isopropyl, butyl, isobutyl, tert-butyl, pentyl or hexyl;
the term “alkylene” is intended to mean a linear or branched, saturated divalent alkyl group; for example, a C₁₋₆-alkylene group is a linear or branched, divalent carbon-based chain of 1 to 6 carbon atoms, for example a methylene, ethylene, 1-methylethylene or propylene;
the term “cycloalkyl” is intended to mean a cyclic alkyl group; for example, a C₃₋₇-cycloalkyl group is a cyclic carbon-based group of 3 to 7 carbon atoms, for example a cyclopropyl, cyclobutyl, cyclopentyl, cyclohexyl or cycloheptyl;
the term “hydroxyl” is intended to mean an —OH group;
the term “cyclic monoamine” is intended to mean a saturated cyclic carbon-based chain comprising one nitrogen atom;
the term “hydroxyalkyl” is intended to mean an alkyl group in which a hydrogen atom has been substituted with a hydroxyl group;
the term “alkyloxy” is intended to mean an —O-alkyl group;
the term “alkylthio” is intended to mean an —S-alkyl group;
the term “fluoroalkyl” is intended to mean an alkyl group in which one or more hydrogen atoms have been substituted with a fluorine atom;
the term “fluoroalkyloxy” is intended to mean an alkyloxy group in which one or more hydrogen atoms have been substituted with a fluorine atom;
the term “a halogen atom” is intended to mean a fluorine, chlorine, bromine or iodine atom;
the term “aryl” is intended to mean a monocyclic or bicyclic aromatic group containing between 6 and 10 carbon atoms. By way of example of an aryl group, mention may be made of phenyl or naphthyl groups.

By way of nonlimiting examples of cyclic amines or diamines formed by N, A, L and B, mention may in particular be made of aziridine, azetidine, pyrrolidine, piperidine, azepine, morpholine, thiomorpholine, homopiperidine, decahydroquinoline, decahydroisoquinoline, azabicycloheptane, azabicyclooctane, azabicyclononane, azaoxobicycloheptane, azathiabicycloheptane, azaoxobicyclooctane, azathiabicyclooctane; piperazine, homopiperazine, diazacyciooctane, diazacyclononane, diazacyclodecane, diazacycloundecane, octahydro-pyrrolopyrazine, octahydropyrrolodiazepine, hexahydropyrrolopyrrole, octahydropyrrolopyridine, decahydronaphthyridine, diazabicycloheptane, diazabicyclooctane, diazabicyclononane, diazaspiroheptane, diazaspirooctane, diazaspirononane, diazaspirodecane, diazaspiroundecane and oxadiazaspiroundecane.

Among the compounds which are subjects of the invention, a first compound group comprises the compounds for which;

R₂ is a thienyl group, optionally substituted with one or more substituents chosen from halogen atoms and C₁₋₆-alkyl groups;

the other substituents being as defined above.

Among the compounds which are subjects of the invention, a second compound group comprises the compounds for which:

R₂ is a thienyl group, optionally substituted with one or more substituents, which may be identical to or different from one another, chosen from a chlorine atom and a methyl group;

the other substituents being as defined above.

Among the compounds which are subjects of the invention, a third compound group comprises the compounds for which:

R₂ is a furanyl group, optionally substituted with one or more substituents, which may be identical to or different from one another, chosen from halogen atoms and C₁₋₆-alkyl groups;

the other substituents being as defined above.

Among the compounds which are subjects of the invention, a fourth compound group comprises the compounds for which;

R₂ is a furanyl group, optionally substituted with one or more C₁₋₆ alkyl groups, more particularly methyl;

the other substituents being as defined above.

Among the compounds which are subjects of the invention, a fifth compound group comprises the compounds for which:

R₂ is a thien-2-yl, 5-methylthien-2-yl, 5-chlorothien-2-yl, thien-3-yl, 2,5-dimethylthien-3-yl, 2,5-dichlorothien-3-yl, furan-2-yl, 5-methylfuran-2-yl or furan-3-yl group;

the other substituents being as defined above.

Among the compounds which are subjects of the invention, a sixth compound group comprises the compounds for which:

R₃ is a hydrogen atom or a C₁₋₃-alkyl or —NR₄R₅ group;

R₄ and R₅ are, independently of one another, a hydrogen atom or a C₁₋₄-alkyl group;

the other substituents being as defined above.

Among the compounds which are subjects of the invention, a seventh compound group comprises the compounds for which;

R₃ is a hydrogen atom, a methyl group or an —NH₂ group;

the other substituents being as defined above.

Among the compounds which are subjects of the invention, an eighth compound group comprises the compounds for which;

R₇ and R₅ are a hydrogen atom;

the other substituents being as defined above.

Among the compounds which are subjects of the invention, a ninth compound group comprises the compounds for which:
A is a C₁₋₇-alkylene group optionally substituted with one or two Rₐ groups;
B is a C₁₋₇-alkylene group optionally substituted with an R_{b} group;
L is either a nitrogen atom optionally substituted with an R_{c} or R_{d} group, or a carbon atom substituted with an Rₑ₁ group and an R_{d} group or two Rₑ₂ groups;

the carbon atoms of A and of B being optionally substituted with one or more R_{f} groups, which may be identical to or different from one another;

Rₐ, R_{b} and R_{c} are defined such that:
two Rₐ groups can together form a C₁₋₆-alkylene group;
Rₐ and R_{b} can together form a bond or a C₁₋₆-alkylene group;
Rₐ and R_{c} can together form a bond or a C₁₋₆-alkylene group;
R_{b} and R_{c} can together form a bond or a C₁₋₆-alkylene group;
R_{d} is a group chosen from a hydrogen atom and C₁₋₆-alkyl and hydroxy-C₁₋₆-alkyl groups;
Rₑ₁ is a cyclic monoamine;
two Rₑ₂ form, with the carbon atom which bears them, a monoamine, this cyclic monoamine being optionally substituted with one or more R_{f} groups, which may be identical to or different from one another;
R_{f} is a C₁₋₆-alkyl group;

the other substituents being as defined above.

Among the compounds which are subjects of the invention, a tenth compound group comprises the compounds for which:

the cyclic amine formed by —N-A-L-B— is a piperazinyl, hexahydropyrrolopyrrolyl, octahydropyrrolopyridinyl, diazaspiroundecyl or pyrrolidinylpiperidinyl group, optionally substituted with one or more groups chosen, independently of one another, from a C₁₋₆-alkyl group and a hydroxy-C₁₋₆-alkyl group;

the other substituents being as defined above.

Among the compounds which are subjects of the invention, an eleventh compound group comprises the compounds for which:

the cyclic amine formed by —N-A-L-B— is a piperazin-1-yl, 3-methylpiperazin-1-yl, 4-methylpiperazin-1-yl, 3,3-dimethylpiperazin-1-yl, (cis)-3,5-dimethylpiperazin-1-yl, 4-(2-hydroxyethyl)piperazin-1-yl, 4-(2-hydroxy-2-methylpropyl)piperazin-1-yl, (cis)-hexahydropyrrolo[3,4-c]pyrrol-2(1H)-yl, (cis)-5-methylhexahydropyrrolo[3,4-c]pyrrol-2(1H)-yl, octahydro-6H-pyrrolo[3,4-b]pyridin-6-yl, 2,9-diazaspiro[5.5]undec-9-yl or 4-pyrrolidin-1-ylpiperidin-1-yl group;

the other substituents being as defined above.

Among the compounds which are subjects of the invention, a twelfth compound group comprises the compounds for which:

R₂ is a thien-2-yl, 5-methylthien-2-yl, 5-chlorothien-2-yl, thien-3-yl, 2,5-dimethylthien-3-yl, 2,5-dichlorothien-3-yl, furan-2-yl, 5-methylfuran-2-yl or furan-3-yl group;

R₃ is a hydrogen atom, a methyl group or an —NH₂ group;

R₇ and R₈ are a hydrogen atom;

the cyclic amine formed by —N-A-L-B— is a piperazin-1-yl, 3-methylpiperazin-1-yl, 4-methylpiperazin-1-yl, 3,3-dimethylpiperazin-1-yl, (cis)-3,5-dimethylpiperazin-1-yl, 4-(2-hydroxyethyl)piperazin-1-yl, 4-(2-hydroxy-2-methylpropyl)piperazin-1-yl, (cis)-hexahydropyrrolo[3,4-c]pyrrol-2(1H)-yl, (cis)-5-methylhexahydropyrrolo[3,4-c]pyrrol-2(1H)-yl, octahydro-6H-pyrrolo[3,4-b]pyridin-6-yl, 2,9-diazaspiro[5.5]undec-9-yl or 4-pyrrolidin-1-yl-piperidin-1-yl group;

the other substituents being as defined above.

Among the compounds of general formula (I) which are subjects of the invention, mention may in particular be made of the following compounds:
6-(piperazin-1-yl)-3-(pyridin-4-yl)-2-(thien-2-yl)imidazo[1,2-b]pyridazine;
3-(2-methylpyridin-4-yl)-6-(piperazin-1-yl)-2-(thien-2-yl)imidazo[1,2-b]pyridazine;
6-(3-methylpiperazin-1-yl)-3-(pyridin-4-yl)-2-(thien-2-yl)imidazo[1,2-b]pyridazine;
2-[4-(3-(pyridin-4-0)-2-(thien-2-yl)imidazo[1,2-b]pyridazin-6-yl)piperazin-1-yl]ethanol;
2-methyl-1-[4-(3-(pyridin-4-yl)-2-(thien-2-yl)imidazo[1,2-b]pyridazin-6-yl)piperazin-1-yl]-propan-2-ol;
6-[(cis)-hexahydropyrrolo[3,4-c]pyrrol-2(1H)-yl]-3-(pyridin-4-yl)-2-(thien-2-yl)imidazo[1,2-b]pyridazine;
6-(octahydropyrrolo[3,4-/]pyridin-6-yl)-3-(pyridin-4-yl)-2-(thien-2-yl)imidazo[1,2-b]pyridazine;
9-(3-(pyridin-4-yl)-2-(thien-2-yl)imidazo[1,2-b]pyridazin-6-yl)-2,9-diazaspiro[5.5]undecane;
3-(pyridin-4-yl)-6-(4-pyrrolidin-1-ylpiperidin-1-yl)-2-(thien-2-yl)imidazo[1,2-b]pyridazine;
2-(5-methylthien-2-yl)-6-(piperazin-1-yl)-3-(pyridin-4-yl)imidazo[1,2-b]pyridazine;
3-(2-methylpyridin-4-yl)-2-(5-methylthien-2-yl)-6-(piperazin-1-yl)imidazo[1,2-b]pyridazine;
4-[2-(5-methylthien-2-yl)-6-(piperazin-1-yl)imidazo[1,2-b]pyridazin-3-yl]pyridin-2-ylamine;
2-(5-chlorothien-2-yl)-6-[(cis)-3,5-dimethylpiperazin-1-yl]-3-(pyridin-4-yl)imidazo[1,2-b]pyridazine;
2-{4-[2-(5-chlorothien-2-yl)-3-(pyridin-4-yl)imidazo[1,2-b]pyridazin-6-yl]piperazin-1-yl}ethanol;
2-(5-chlorothien-2-yl)-6-[(cis)-5-methylhexahydropyrrolo[3,4-c]pyrrol-2(1H)-yl]-3-(pyridin-4-yl)imidazo[1,2-b]pyridazine;
2-(5-chlorothien-2-yl)-6-(octahydro-6H-pyrrolo[3,4-b]pyridin-6-0)-3-(pyridin-4-yl)imidazo[1,2-b]pyridazine;
4-(6-(piperazin-1-yl)-2-(thien-3-yl)imidazo[1,2-b]pyridazin-3-yl)pyridin-2-ylamine;
6-(4-methylpiperazin-1-yl)-3-(pyridin-4-yl)-2-(thien-3-yl)imidazo[1,2-b]pyridazine;
2-methyl-1-[4-(3-(pyridin-4-yl)-2-(thien-3-yl)imidazo[1,2-b]pyridazin-6-yl)piperazin-1-yl]propan-2-ol;
6-[(cis)-hexahydropyrrolo[3,4-c]pyrrol-2-yl]-3-(pyridin-4-yl)-2-(thien-3-yl)imidazo[1,2-b]pyridazine;
6-(octahydro-6H-pyrrolo[3,4-b]pyridin-6-yl)-3-(pyridin-4-yl)-2-(thien-3-yl)imidazo[1,2-b]pyridazine and the trihydrochloride thereof;
9-[3-(pyridin-4-yl)-2-(thien-3-yl)imidazo[1,2-b]pyridazin-6-yl]-2,9-diazaspiro[5.5]undecane;
3-(pyridin-4-yl)-6-(4-pyrrolidin-1-ylpiperidin-1-yl)-2-(thien-3-yl)imidazo[1,2-b]pyridazine;
2-(2,5-dimethylthien-3-0)-6-(piperazin-1-yl)-3-(pyridin-4-yl)imidazo[1,2-b]pyridazine;
2-(2,5-dimethylthien-3-yl)-3-(2-methylpyridin-4-yl)-6-(piperazin-1-yl)imidazo[1,2-b]pyridazine;
4-[2-(2,5-dimethylthien-3-yl)-6-(piperazin-1-yl)imidazo[1,2-b]pyridazin-3-yl]pyridin-2-ylamine;
2-(2,5-dichlorothien-3-O-6-(3,3-dimethylpiperazin-1-yl)-3-(pyridin-4-yl)imidazo[1,2-b]-pyridazine;
2-{4-[2-(5-methylfuran-2-yl)-3-pyridin-4-ylimidazo[1,2-b]pyridazin-6-yl]piperazin-1-yl}ethanol;
2-methyl-1-{4-[2-(5-methylfuran-2-yl)-3-pyridin-4-ylimidazo[1,2-b]pyridazin-6-yl]piperazin-1-yl}propan-2-ol;
2-[4-(2-furan-3-O-3-pyridin-4-ylimidazo[1,2-b]pyridazin-6-yl)piperazin-1-yl]ethanol;
1-[4-(2-furan-3-yl-3-pyridin-4-ylimidazo[1,2-b]pyridazin-6-yl)piperazin-1-yl]-2-methylpropan-2-ol;
2-(furan-2-yl)-6-[(cis)-5-methylhexahydropyrrolo[3,4-c]pyrrol-2(1H)-yl]-3-(pyridin-4-yl)imidazo[1,2-b]pyridazine,
2-(5-methylfuran-2-yl)-6-[(cis)-5-methylhexahydropyrrolo[3,4-c]pyrrol-2(1H)-yl]-3-pyridin-4-ylimidazo[1,2-b]pyridazine;
2-furan-3-yl-6-[(cis)-5-methylhexahydropyrrolo[3,4-c]pyrrol-2(1H-yl]-3-pyridin-4-ylimidazo[1,2-b]pyridazine.

In accordance with the invention, the compounds of general formula (I) can be prepared according to the general process described in scheme 1 below.

In general and as illustrated in scheme 1, the 6-cycloamino-3-(pyridin-4-yl)imidazo-[1,2-b]pyridazine derivatives of general formula (I) in which R₂, R₃, A, L, B, R₇ and R₈ are as defined above, can be prepared from a 3-(pyridin-4-yl)imidazo[1,2-b]pyridazine derivative of general formula (II), in which R₂, R₃, R₇ and R₈ are as defined above and X₆ is a leaving group such as a halogen, by treatment with an amine of general formula (IIa) in which A, L and B are as defined above. This reaction can be carried out by heating the reactants in a polar solvent such as pentanol or dimethyl sulphoxide.

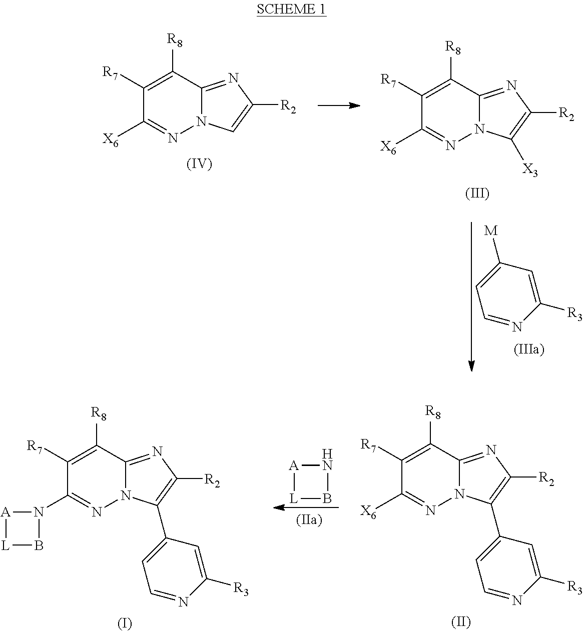

The 3-(pyridin-4-yl)imidazo[1,2-b]pyridazine derivatives of general formula (II), in which R₂, R₃, X₆, R₇ and R₈ are as defined above, can be prepared by metal-catalysed coupling of a 3-haloimidazo[1,2-b]pyridazine derivative of general formula (III) in which R₂, X₆, R₇ and R₈ are as defined above and X₃ is a halogen chosen from bromine and iodine, more particularly iodine, with a pyridine derivative of general formula (IIIa) in which R₃ is as defined above and M is a trialkylstannyl group, most commonly a tributylstannyl group or a dihydroxyboryl or dialkyloxyboryl group, most commonly a 4,4,5,5-tetramethyl-1,3,3,2-dioxaborolan-2-yl group, according to Stille or Suzuki conditions.

The couplings according to the Stifle method are, for example, performed by heating, in the presence of a catalyst such as tetrakis(triphenylphosphine)palladium, copper iodine, in a solvent such as N,N-dimethylacetamide.

The couplings according to the Suzuki method are, for example, performed by heating, in the presence of a catalyst such as [1,1′-bis(diphenylphosphino)ferrocene]dichloropalladium, of a mineral base such as caesium carbonate, in a mixture of solvents such as dioxane and water.

The 3-haloimidazo[1,2-b]pyridazine derivatives of general formula (III) are obtained by regioselective bromination or iodination of an imidazo[1,2-b]pyridazine derivative of general formula (IV), in which R₂, X₆, R₇ and R₈ are as defined above. This reaction can be carried out by means of N-bromo- or iodosuccinimide or iodine monochloride in a polar solvent such as acetonitrile, tetrahydrofuran, methanol or chloroform.

The imidazo[1,2-b]pyridazine derivatives of general formula (IV) are known to those skilled in the art (Journal of Heterocyclic Chemistry (2002), 39(4), 737-742) or can be prepared by analogy with methods known to those skilled in the art.

Alternatively, and according to scheme 2, the 6-cycloamino-3-pyridin-4-ylimidazo-[1,2-b]pyridazine derivatives of general formula (I) in which R₂, R₃, A, L, B, R₇ and R₈ are as defined above, can be prepared by metal-catalysed coupling between a 3-haloimidazo[1,2-b]pyridazine derivative of general formula (V) in which R₂, A, L, B, R₇ and R₈ are as defined above and X₃ is a halogen chosen from bromine and iodine, more particularly iodine, and a pyridine derivative of general formula (IIIa) as defined above, according to Stille or Suzuki conditions.

The 3-haloimidazo[1,2-b]pyridazine derivatives of general formula (V) are obtained by regioselective bromination or iodination of an imidazo[1,2-b]pyridazine derivative of general formula (VI), in which R₂, A, L, B, R₇ and R₈ are as defined above. This reaction can be carried out by means of N-bromo- or iodosuccinimide or iodine monochloride, in a polar solvent such as acetonitrile, tetrahydrofuran, methanol or chloroform.

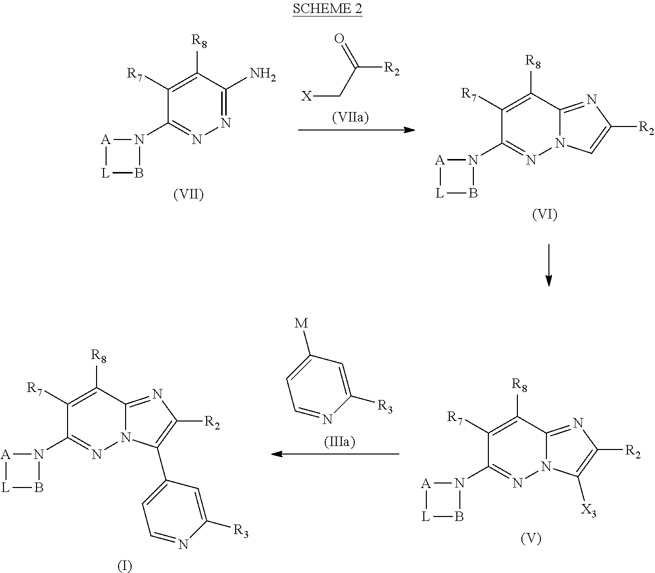

The 3-pyridin-4-ylimidazo[1,2-b]pyridazine derivatives of general formula (VI) in which R₂, A, L, B, R₇ and R₈ are as defined above, are prepared by condensation between a pyridazin-3-ylamine derivative of general formula (VII), in which A, L, B, R₇ and R₈ are as defined above and a 2-bromo-, chloro- or iodoethan-1-one derivative of general formula (VIIa) in which R₂ is as defined above and X is a bromine, chlorine or iodine atom.

The reaction can be carried out by heating the reactants in a polar solvent such as ethanol or butanol.

The pyridazin-3-ylamine derivatives of general formula (VII) are known to those skilled in the art (Journal of Medicinal Chemistry (2008), 51(12), 3507-3525) or can be prepared by analogy with methods known to those skilled in the art.

Specifically, according to scheme 3, the 6-cycloamino-3-pyridin-4-ylimidazo[1,2-b]pyridazine derivatives of general formula (I) in which R₂, A, L, B, R₇ and R₈ are as defined above and in which R₃ is a hydrogen atom or a C₁₋₃-alkyl group, can be prepared, in two stages, from an imidazo[1,2-b]pyridazine derivative of general formula (VI) as defined above.

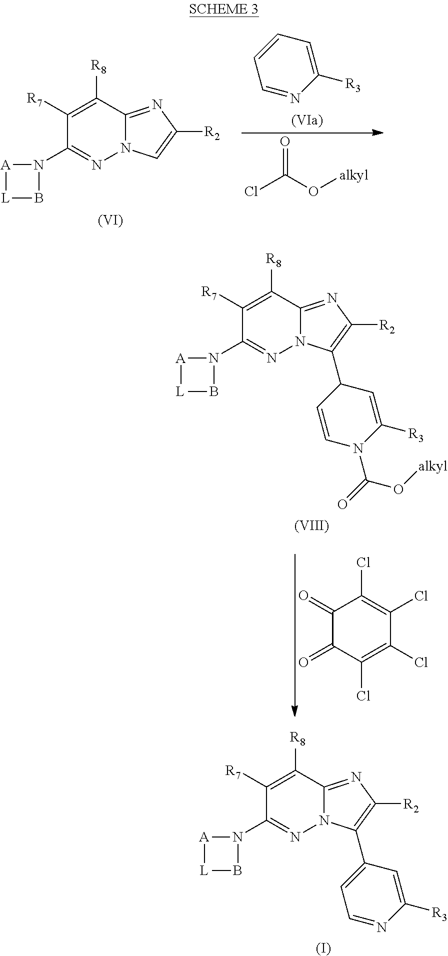

Thus, the reaction of an imidazo[1,2-b]pyridazine derivative of general formula (VI) with a mixture of a pyridine derivative of general formula (VIa), in which R₃ is a hydrogen atom or a C₁₋₃-alkyl group, and of alkyl chloroformate in which the alkyl group is a C₁₋₆-alkyl, for example ethyl chloroformate, leads to the derivative of general formula (VIII) in which R₂, A, L, B, R₇ and R₈ are as defined above and in which R₃ is a hydrogen atom or a C₁₋₃-alkyl group. The derivative of general formula (VIII) is then oxidized using ortho-chloranil in a solvent such as toluene, to give the 6-cycloamino-3-pyridin-4-ylimidazo[1,2-b]pyridazine derivatives of general formula (I) in which R₂, A, L, B, R₇ and R₈ are as defined above and in which R₃ is a hydrogen atom or a C₁₋₃-alkyl group.

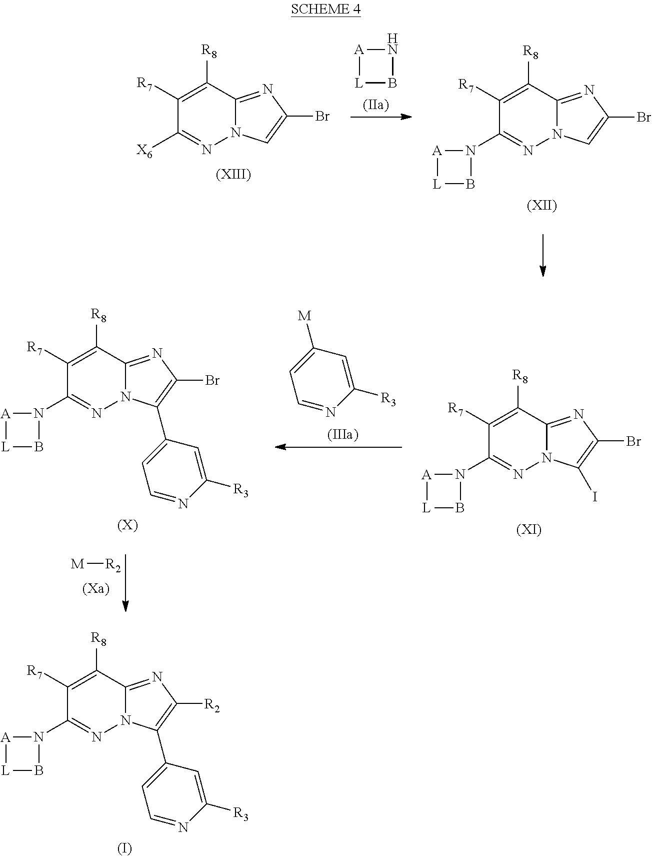

Finally, and according to scheme 4, the 6-cycloamino-3-pyridin-4-ylimidazo[1,2-b]pyridazine derivatives of general formula (I) in which R₂, R₃, A, L, B, R₇ and R₈ are as defined above, can be prepared by metal-catalysed coupling according to Stille or Suzuki conditions as defined above, between a 2-bromo-3-pyridinimidazo[1,2-b]pyridazine derivative of general formula (X), in which R₃, A, L, B, R₇ and R₈ are as defined above, and a thienyl or furanyl derivative, of general formula (Xa) where R₂ and M are as defined above.

The 2-bromo-3-pyridinimidazo[1,2-b]pyridazine derivatives of general formula (X) are obtained by regioselective metal-catalysed coupling according to Stille or Suzuki conditions as defined above, between a 2-bromo-3-iodoimidazo[1,2-b]pyridazine derivative of general formula (XI), in which A, L, B, R₇ and R₈ are as defined above, and a pyridine derivative of general formula (IIIa) as defined above.

The 2-bromo-3-iodoimidazo[1,2-b]pyridazine derivatives of general formula (XI) are obtained by iodination of a 2-bromoimidazo[1,2-b]pyridazine derivative of general formula (XII), in which A, L, B, R₇ and R₈ are as defined above. This reaction can be carried out by means of N-iodosuccinimide or of iodine monochloride, in a polar solvent such as acetonitrile, tetrahydrofuran, methanol or chloroform.

The 2-bromoimidazo[1,2-b]pyridazine derivatives of general formula (XII) are obtained from a 2-bromoimidazo[1,2-b]pyridazine derivative of general formula (XIII), in which R₇ and R₈ are as defined above and X₈ is a leaving group such as a halogen, by treatment with an amine of general formula (IIa), in which A, L and B are as defined above. This reaction can be carried out by heating the reactants in a polar solvent such as pentanol or dimethyl sulphoxide.

The 2-bromoimidazo[1,2-b]pyridazine derivatives of general formula (XIII) are known to those skilled in the art or can be prepared by analogy with methods described in the literature (WO2009/037394).

In certain cases, the 6-cycloamino-3-(pyridin-4-yl)imidazo[1,2-b]pyridazine derivatives of general formula (I), for which the amine formed by N. L. A and B comprises a second, secondary or tertiary amine, can be prepared, respectively, from the corresponding primary or secondary amine by alkylation or reductive amination according to methods customary for those skilled in the art.

In the preceding text, the term “leaving group” is intended to mean a group that can be readily cleaved from a molecule by heterolytic bond breaking, with the departure of a pair of electrons. This group can, for example, thus be readily replaced with another group in a substitution reaction. Such leaving groups are, for example, halogens or an activated hydroxyl group such as a mesyl, tosyl, triflate, acetyl, etc. Examples of leaving groups and also references for the preparation thereof are given in “Advances in Organic Chemistry”, J. March, 3^{rd} Edition, Wiley Interscience, p. 310-316,

### Protecting Groups

For the compounds of general formula (I) or (IIa) as defined above and in the case where the N-A-L-B group comprises a primary or secondary amine function, this function may optionally be protected, during the synthesis, with a protecting group, for example a benzyl or a t-butyloxycarbonyl.

The following examples describe the preparation of some compounds in accordance with the invention. These examples are not limiting and serve merely to illustrate the invention. The numbers of the compounds exemplified refer back to those given in Table 1 hereinafter, which illustrate the chemical structures and the physical properties, respectively, of a number of compounds according to the invention.

### EXAMPLE NO. 1

### (Compound No. 1): 6-(piperazin-1-yl)-3-(pyridin-4-yl)-2-(thien-2-yl)imidazo[1,2-b]pyridazine

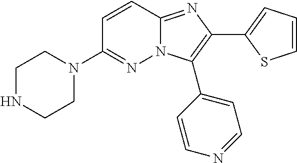 [SMILES: c1csc(-c2nc3ccc(N4CCNCC4)nn3c2-c2ccncc2)c1]

### Stage 1.1. 6-(piperazin-1-yl)pyridazin-3-ylamine

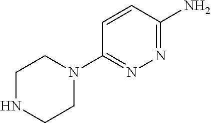 [SMILES: Nc1ccc(N2CCNCC2)nn1]

A mixture of 2.00 g (15.4 mmol) of 3-amino-6-chloropyridazine and 8.8 g (77 mmol) of piperazine-1-carbaldehyde is heated at 140° C. for 5 hours. After cooling, the mixture is chromatographed on an alumina column, elution being carried out with a mixture of dichloromethane and methanol (98/2), to give 1.2 g of product in the form of a yellow solid after trituration in diethyl ether and drying.

1.0 g (4.8 mmol) of the solid obtained is solubilized in 5 ml of tetrahydrofuran and is treated with 18 ml (72 mmol) of 4N aqueous sulphuric acid at 80° C. for 2 hours. The medium is neutralized by adding a saturated solution of sodium hydrogen carbonate. The solvent is evaporated off under reduced pressure, the residue is triturated with chloroform and the solution is filtered. The filtrate is concentrated under reduced pressure and the residue is chromatographed on a silica gel column, elution being carried out with a mixture of dichloromethane, methanol and aqueous ammonia (90/10/1), to give 0.53 g of 6-(piperazin-1-yl)pyridazin-3-ylamine in the form of a brown oil which crystallizes.

¹H NMR (CDCl₃) δ: 6.90 (d, 1H); 6.70 (d, 1H); 4.2 (broad signal, 2H); 3.4 (m, 4H); 3.00 (m, 4H) ppm.

### Stage 1.2. tert-Butyl 4-(6-aminopyridazin-3-yl)piperazine-1-carboxylate

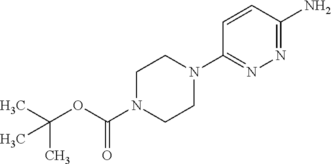 [SMILES: CC(C)(C)OC(=O)N1CCN(c2ccc(N)nn2)CC1]

0.41 ml (2.9 mmol) of triethylamine and 0.64 g (2.9 mmol) of di-tert-butyl dicarbonate are added to a solution, cooled to 0° C., of 0.52 g (2.9 mmol) of piperazin-1-ylpyridazin-3-ylamine in 10 ml of tetrahydrofuran. The mixture is stirred for 1 hour and is left to return to ambient temperature, and then 100 ml of water are added and the product is extracted with dichloromethane. The organic solution is separated on a hydrophobic filtration cartridge and the solvent is evaporated off under reduced pressure. 0.48 g of tert-butyl 4-(6-amino-pyridazin-3-yl)piperazine-1-carboxylate is isolated in the form of a yellow powder after crystallization from diisopropyl ether and drying.

¹H NMR (CDCl₃) δ: 7.00 (d, 1H); 6.80 (d, 1H); 4.4 (broad signal, 2H); 3.6 (m, 4H); 3.5 (m, 4H); 1.55 (s, 9H) ppm.

### Stage 1.3. tert-Butyl 4-(2-(thien-2-yl)imidazo[1,2-b]pyridazin-6-yl)piperazine-1-carboxylate

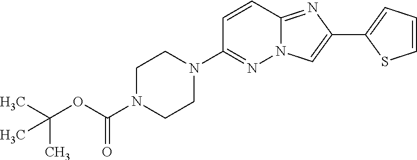 [SMILES: CC(C)(C)OC(=O)N1CCN(c2ccc3nc(-c4cccs4)cn3n2)CC1]

0.88 g (4.3 mmol) of 2-bromo-1-(thien-2-yl)ethanone is added to a solution, heated to 100° C., of 1.00 g (3.58 mmol) of tert-butyl 4-(6-aminopyridazin-3-yl)piperazine-1-carboxylate in 100 ml of n-butanol. The mixture is stirred for 30 minutes and is poured into a saturated aqueous solution of sodium hydrogen carbonate and the product is extracted with dichloromethane. The organic solution is separated and dried over sodium sulphate and the solvent is evaporated off under reduced pressure. 1.2 g of product are isolated in the form of a yellow solid after rinsing in petroleum ether.

Said product is purified by silica gel column chromatography, elution being carried out with a mixture of dichloromethane, methanol and aqueous ammonia (95/5/0.5), to give 1.0 g of tert-butyl 4-(2-(thien-2-yl)imidazo[1,2-b]pyridazin-6-yl)piperazine-1-carboxylate in the form of a beige solid.

Mp 165-167° C.

¹H NMR (CDCl₃) δ: 7.90 (d, 1H); 7.70 (d, 1H); 7.40 (m, 1H); 7.30 (m, 1H); 7.10 (m, 1H); 6.80 (d, 1H); 3.6 (m, 4H); 3.5 (m, 4H); 1.55 (s, 9H) ppm.

Stage 1.4. tert-Butyl 4-[3-(1-ethoxycarbonyl-1,4-dihydropyridin-4-yl)-2-(thien-2-yl)-imidazo[1,2-b]pyridazin-6-yl]piperazine-1-carboxylate

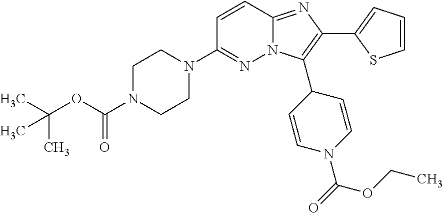 [SMILES: CCOC(=O)N1C=CC(c2c(-c3cccs3)nc3ccc(N4CCN(C(=O)OC(C)(C)C)CC4)nn23)C=C1]

2.6 ml (51 mmol) of ethyl chloroformate are added, under argon and dropwise, to a suspension, cooled to 0° C., of 1.04 g (2.70 mmol) of tert-butyl 4-(2-(thien-2-yl)imidazo[1,2-b]pyridazin-6-yl)piperazine-1-carboxylate in 8.7 ml of pyridine, while maintaining the temperature at 0° C. The heterogeneous medium is subsequently allowed to return to ambient temperature. After stirring for 2 and a half hours, the suspension is again cooled to 0° C. and 2.6 ml (51 mmol) of ethyl chloroformate are again added. After the addition, the reaction is allowed to return to ambient temperature and the reaction is left for 18 hours. The mixture is diluted with dichloromethane and is poured into water. The organic phase is separated and dried over sodium sulphate and the solvent is removed by evaporation under reduced pressure. The brown solid obtained (1.4 g) is recrystallized from approximately 30 ml of acetonitrile, to give 1.10 g of tert-butyl 4-[3-(1-ethoxycarbonyl-1,4-dihydropyridin-4-yl)-2-(thien-2-yl)imidazo[1,2-b]pyridazin-6-yl]piperazine-1-carboxylate in the form of a solid after filtration, rinsing with diethyl ether and drying.

Mp 155° C.

¹H NMR (CDCl₃) δ: 7.75 (d, 1H); 7.45 (m, 2H); 7.10 (dd, 1H); 7.3 (md, 2H); 6.80 (d, 1H); 5.25 (m, 1H); 4.9 (m, 2H); 4.35 (q, 2H); 3.55 (m, 4H); 3.45 (m, 4H); 1.50 (s, 9H); 1.40 (t, 3H) ppm.

### Stage 1.5. tert-Butyl 4-(3-(pyridin-4-yl)-2-(thien-2-yl)imidazo[1,2-b]pyridazin-6-yl)piperazine-1-carboxylate

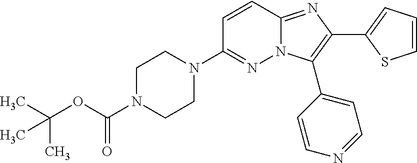 [SMILES: CC(C)(C)OC(=O)N1CCN(c2ccc3nc(-c4cccs4)c(-c4ccncc4)n3n2)CC1]

0.554 g (2.25 mmol) of ortho-chloranil in solution in 15 ml of toluene is added to a solution of 1.10 g (2.05 mmol) of tert-butyl 4-[3-(1-ethoxycarbonyl-1,4-dihydropyridin-4-yl)-2-(thien-2-yl)imidazo[1,2-b]pyridazin-6-yl]piperazine-1-carboxylate in 50 ml of toluene. After stirring for 1 h, the solution is poured into a saturated aqueous solution of sodium hydroxide and the product is extracted with dichloromethane. The organic phase is dried over sodium sulphate and concentrated under reduced pressure, to give 1.1 g of an amorphous solid. The latter is purified by silica gel column chromatography, elution being carried out with a mixture of dichloromethane, methanol and aqueous ammonia (94/4/0.4), to give 0.67 g of tert-butyl 4-(3-(pyridin-4-yl)-2-(thien-2-yl)imidazo[1,2-b]yl)pyridazin-6-yl)piperazine-1-carboxylate in the form of a pale yellow solid, after crystallization from diethyl ether and drying.

Mp 223-226° C.

¹H NMR (CDCl₃) δ: 8.80 (d, 2H); 7.90 (d, 1H); 7.85 (d, 2H); 7.45 (d, 1H); 7,25/d. 1H);

### Stage 1.6. 6-(piperazin-1-yl)-3-(pyridin-4-yl)-2-(thien-2-y)imidazo[1,2-b]pyridazine

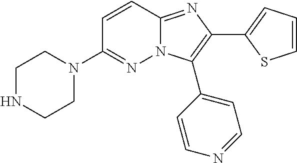 [SMILES: c1csc(-c2nc3ccc(N4CCNCC4)nn3c2-c2ccncc2)c1]

2.2 ml of trifluoroacetic acid are added slowly to a solution of tert-butyl 4-(3-(pyridin-4-yl)-2-(thien-2-yl)imidazo[1,2-b]pyridazin-6-yl)piperazine-1-carboxylate in 35 ml of dichloromethane cooled to 0° C., and the solution is stirred at ambient temperature for 2 hours. The solution is then poured into an aqueous solution of sodium hydroxide, the organic phase is separated and the aqueous phase is washed with dichloromethane. The organic phases are dried over sodium sulphate and concentrated under reduced pressure. The solid obtained is purified by silica gel column chromatography, elution being carried out with a mixture of dichloromethane, methanol and aqueous ammonia (92/8/0.8), to give 0.47 g of a pale yellow solid. 0.36 g of 6-(piperazin-1-yl)-3-(pyridin-4-yl)-2-(thien-2-yl)imidazo[1,2-b]pyridazine is isolated after crystallization from 20 ml of acetonitrile containing a few ml of butanol, and then drying.

Mp 217-220° C.

¹H NMR (CDCl₃) δ: 8.75 (d, 2H); 7.80 (d, 2H); 7.70 (d, 2H); 7.35 (dd, 1H); 7.20 (dd, 1H); 7.00 (dd, 1H); 6.90 (m, 11-1); 3.50 (m, 4H); 3.0 (m, 4H); 2.90 (sl, 1H) ppm.

### EXAMPLE NO. 2

### (Compound No. 9): 3-(Pyridin-4-yl)-6-(4-pyrrolidin-1-ylpiperidin-1-yl)-2-(thien-2-yl)imidazo[1,2-b]pyridazine

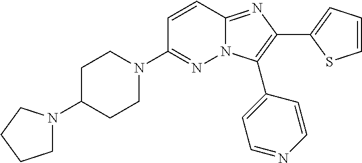 [SMILES: c1csc(-c2nc3ccc(N4CCC(N5CCCC5)CC4)nn3c2-c2ccncc2)c1]

### Stage 2.1. 6-Chloro-2-(thien-2-yl)imidazo[1,2-b]pyridazine

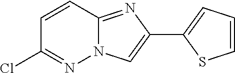 [SMILES: Clc1ccc2nc(-c3cccs3)cn2n1]

5.00 g (24.4 mmol) of 2-bromo-1-(thien-3-yl)ethanone are added portionwise to a solution of 2.63 g (20.3 mmol) of 3-amino-6-chloropyridazine in 150 ml of butanol, and the mixture is heated at 90° C. for 3 hours. After cooling, the solvent is evaporated off under reduced pressure, the residue is taken up with chloroform and the solution is neutralized with an aqueous solution of sodium hydroxide. The organic phase is separated and dried over sodium sulphate, to give a brown solid after evaporation of the solvent. The solid is triturated in a mixture of 75 ml of isopropanol and diisopropyl ether (1/1), to give 2.69 g of 6-chloro-2-(thien-2-yl)imidazo[1,2-b]pyridazine in the form of a dark beige solid, after filtration and drying under reduced pressure.

Mp 223-225° C.

¹H NMR (DMSOd₆) δ: 8.15 (s, 1H); 7.90 (d, 1H); 7.50 (d, 1H); 7.40 (d, 1H); 7.15 (dd, 1H); 7.05 (d, 1H) ppm.

### Stage 2.2. 6-Chloro-3-iodo-2-(thien-2-yl)imidazo[1,2-b]pyridazine

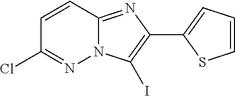 [SMILES: Clc1ccc2nc(-c3cccs3)c(I)n2n1]

20.4 ml (20.4 mmol) of a 1M solution of iodine chloride in dichloromethane are added, at ambient temperature, to a solution of 2.45 g (10.4 mmol) of 6-chloro-2-(thien-2-yl)-imidazo[1,2-b]pyridazine in 200 ml of chloroform. After reaction for 20 minutes, a further 20.4 ml (20.4 mmol) of a 1M solution of iodine chloride in dichloromethane are added and the reaction is continued for 15 minutes. The solution is then poured into a saturated solution of potassium bicarbonate and the mixture is decoloured by adding a 5% aqueous solution of sodium thiosulphate. The organic phase is separated, dried over sodium sulphate and concentrated under reduced pressure, to give a yellowish solid, which is purified by silica gel column chromatography, elution being carried out with dichloromethane, to give 2.24 g of 6-chloro-3-iodo-2-(thien-2-yl)imidazo[1,2-b]pyridazine in the form of a yellow solid.

Mp 205-209° C.

¹H NMR (DMSOd₆) δ: 8.05 (dd, 1H); 7.85 (d, 1H); 7.45 (dd, 1H); 7.20 (dd, 1H); 7.15 (d, 1H) ppm.

### Stage 2.3. 6-Chloro-3-pyridin-4-y-2-(thien-2-yl)imidazo[1,2-b]pyridazine

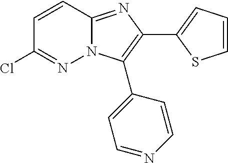 [SMILES: Clc1ccc2nc(-c3cccs3)c(-c3ccncc3)n2n1]

6.7 g (21 mmol) of caesium carbonate and 0.50 g (0.61 mmol) of a complex of [1,1′-bis(diphenylphosphino)ferrocene]dichloropalladium(II) and dichloromethane (PdCl₂(dppf).CH₂Cl₂) are added, after degassing using argon, to a mixture of 2.46 g (6.82 mmol) of 6-chloro-3-iodo-2-(thien-2-yl)imidazo[1,2-b]pyridazine and 1.67 g (8.18 mmol) of 4-(4,4,5,5-tetramethyl-1,3,2-dioxoborolan-2-yl)pyridine in 32 ml of a mixture of tetrahydrofuran and water (9/1). The reaction is stirred at reflux for 18 hours. The mixture is poured into 350 ml of a 1N aqueous solution of hydrochloric acid and the aqueous phase is washed with ethyl acetate. The aqueous phase is then basified using aqueous ammonia and the product is extracted with chloroform. The organic phase is dried over sodium sulphate and the solvent is evaporated off under reduced pressure. The residue is purified by chromatography on a 50 g silica gel column, elution being carried out with a mixture of dichloromethane, methanol and aqueous ammonia (97/3/0.3), to give 1.5 g of 6-chloro-3-(pyridin-4-yl)-2-(thien-2-yl)imidazo[1,2-b]pyridazine in the form of a yellow solid.

Mp: 208-210° C.

¹H NMR (CDCl₃) δ: 8.80 (d, 2H); 8.05 (d, 1H); 7.75 (d, 2H); 7.55 (d, 1H); 7.30 (m, 1H); 7.20 (d, 1H); 7.10 (dd, 1H) ppm.

### Stage 2.4. 3-(Pyridin-4-yl)-6-(4-pyrrolidin-1-ylpiperidin-1-yl)-2-(thien-2-yl)imidazo-[1,2-b]pyridazine

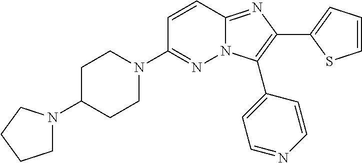 [SMILES: c1csc(-c2nc3ccc(N4CCC(N5CCCC5)CC4)nn3c2-c2ccncc2)c1]

A mixture of 0.25 g (0.80 mmol) of 6-chloro-3-(pyridin-4-yl)-2-(thien-2-yl)imidazo-[1,2-b]pyridazine, 0.37 g (2.4 mmol) of 4-pyrrolidin-1-ylpiperidine and 0.13 ml of diisopropylethylamine in 5 ml of pentanol is refluxed for 18 hours at 140° C. After cooling, the mixture is poured into a 1N aqueous solution of hydrochloric acid and the aqueous phase is washed with ethyl acetate. The aqueous phase is then basified using aqueous ammonia and the product is extracted with chloroform. The organic phase is dried over sodium sulphate and the solvent is evaporated off under reduced pressure. The residue is purified by silica gel column chromatography, elution being carried out with a mixture of dichloromethane, methanol and aqueous ammonia (95/5/0.5), to give 0.26 g of 3-(pyridin-4-yl)-6-(4-pyrrolidin-1-yl-piperidin-1-yl)-2-(thien-2-yl)imidazo[1,2-b]pyridazine in the form of a beige powder after crystallization from 15 ml of acetonitrile, filtration and drying.

Mp: 85° C. (transformation)

¹H NMR (CDCl₃) δ: 8.65 (d, 2H); 7.70 (d, 1H); 7.60 (d, 2H); 7.25 (d, 1H); 7.10 (d, 1H); 6.95 (dd, 1H); 6.85 (d, 1H); 5.95 (d, 2H); 2.9 (t, 2H); 2.55 (m, 4H); 2.12 (m, 1H); 1.95 (m, 2H); 1.75 (m, 4H); 1.5 (m, 2H) ppm.

### EXAMPLE NO. 3

### (Compound No. 5): 2-Methyl-1-[4-(3-(pyridin-4-yl)-2-(thien-2-yl)-imidazo[1,2-b]pyridazin-6-yl)(piperazin-1-yl)]propan-2-ol

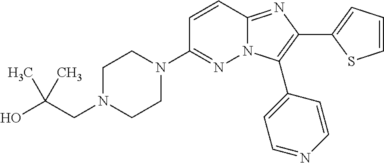 [SMILES: CC(C)(O)CN1CCN(c2ccc3nc(-c4cccs4)c(-c4ccncc4)n3n2)CC1]

A mixture of 0.25 g (0.80 mmol) of 6-chloro-3-(pyridin-4-yl)-2-(thien-2-yl)imidazo-[1,2-b]pyridazine, 0.38 g (2.4 mmol) of 2-methyl-1-[piperazin-1-yl]propan-2-ol and 0.13 ml (0.80 mmol) of diisopropylethylamine in 5 ml of pentanol is refluxed for 18 hours at 140° C. The reaction medium is then cooled and the mixture is poured into a 1N aqueous solution of hydrochloric acid and the aqueous phase is washed with ethyl acetate. The aqueous phase is then basified using aqueous ammonia and the product is extracted with dichloromethane. The organic phase is dried over sodium sulphate and the solvent is evaporated off under reduced pressure. The residue is purified by silica gel column chromatography, elution being carried out with a mixture of dichloromethane, methanol and aqueous ammonia (95/5/0.5), to give 0.19 g of 2-methyl-1-[4-(3-(pyridin-4-yl)-2-(thien-2-yl)imidazo[1,2-b]pyridazin-6-yl)piperazin-1-yl]propan-2-ol in the form of a beige powder after crystallization from 15 ml of acetonitrile, filtration and drying.

Mp: 165-168° C.

¹H NMR (CDCl₃) δ: 8.75 (d, 2H); 7.80 (d, 1H); 7.70 (d, 2H); 7.35 (d, 1H); 7.20 (d, 1H); 7.00 (dd, 1H); 6.90 (d, 1H); 3.50 (d, 4H); 2.8 (m, 5H); 2.50 (s, 2H); 1.25 (s, 6H) ppm.

### EXAMPLE NO. 4

### (Compound No. 7): 6-(Octahydro-6H-pyrrolo[3,4-b]pyridin-6-yl)-3-(pyridin-4-yl)-2-(thien-2-yl)imidazo[1,2-b]pyridazine

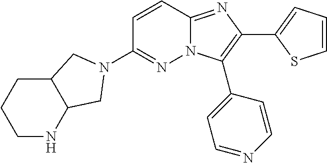 [SMILES: c1csc(-c2nc3ccc(N4CC5CCCNC5C4)nn3c2-c2ccncc2)c1]

A mixture of 0.30 g (0.96 mmol) of 6-chloro-3-(pyridin-4-yl)-2-(thien-2-yl)imidazo-[1,2-b]pyridazine, 0.65 g (2.9 mmol) of tert-butyl 1H-octahydropyrrolo[3,4-b]pyridine-1-carboxylate (CAS 159877-36-8) and 0.16 ml (0.96 mmol) of diisopropylethylamine in 5 ml of pentanol is refluxed for 18 hours at 150° C. The reaction medium is cooled and 5 ml of 3N aqueous hydrochloric acid (15 mmol) are added. The mixture is stirred for one hour and then diluted with water. The aqueous phase is washed with ethyl acetate and then basified using aqueous ammonia, and the product is extracted with dichloromethane. The organic phase is dried over sodium sulphate and the solvent is evaporated off under reduced pressure. The residue is purified by silica gel column chromatography, elution being carried out with a mixture of dichloromethane, methanol and aqueous ammonia (94/6/0.6), to give 0.186 g of 6-(octahydro-6H-pyrrolo[3,4-b]pyridin-6-yl)-3-(pyridin-4-yl)-2-(thien-2-yl)imidazo[1,2-b]pyridazine in the form of a whitish powder after crystallization from 35 ml of diethyl ether, filtration and drying.

Mp: 176-179° C.

¹H NMR (CDCl₃) δ: 8.70 (d, 2H); 7.75 (m, 3H); 7.35 (d, 1H); 7.20 (d, 1H); 7.00 (dd, 1H); 6.65 (d, 1H); 3.5 (m, 5H); 3.05 (m, 1H); 2.70 (m, 1H); 2.40 (s, 1H); 1.9-1.5 (m, 5H) ppm.

### EXAMPLE NO. 5

### (Compound No. 14): 2-{4-[2-(5-Chlorothien-2-yl)-3-(pyridin-4-yl)-imidazo[1,2-b]pyridazin-6-yl](piperazin-1-yl}}methanol

### Stage 5.1. 6-Chloro-2-(5-chlorothien-2-yl)imidazo[1,2-b]pyridazine

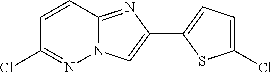 [SMILES: Clc1ccc2nc(-c3ccc(Cl)s3)cn2n1]

A solution of 6.76 g (52.2 mmol) of 3-amino-6-chloropyridazine and 15.0 g (62.6 mmol) of 2-bromo-1-(5-chlorothien-2-yl)ethanone, added portionwise to 280 ml of ethanol, is refluxed for 3 hours. After cooling, the solvent is evaporated off under reduced pressure, the orangey-yellow residue is taken up with chloroform and the solution is neutralized with an aqueous ammonia solution. The organic phase is separated and dried over sodium sulphate, to give a brown solid after evaporation of the solvent. The solid is triturated in 100 ml of acetonitrile, to give 6.0 g of 6-chloro-2-(5-chlorothien-2-yl)imidazo[1,2-b]pyridazine in the form of a dark beige solid after filtration and drying under reduced pressure.

Mp 226-230° C.

¹H NMR (DMSOd₆) δ: 8.80 (s, 1H); 8.20 (d, 1H); 7.50 (d, 1H); 7.40 (d, 1H); 7.20 (d, 1H) ppm.

### Stage 5.2. 6-Chloro-3-iodo-2-(5-chlorothien-2-yl)imidazo[1,2-b]pyridazine and 6-chloro-3-chloro-2-(5-chlorothien-2-yl)imidazo[1,2-b]pyrazine

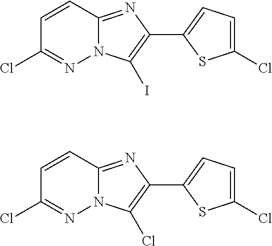 [SMILES: Clc1ccc2nc(-c3ccc(Cl)s3)c(I)n2n1 ; Clc1ccc2nc(-c3ccc(Cl)s3)c(Cl)n2n1]

28.9 ml (28.9 mmol) of a 1M solution of iodine chloride in dichloromethane are added, at ambient temperature, to a solution of 4.30 g (15.9 mmol) of 6-chloro-2-(5-chlorothien-2-yl)imidazo[1,2-b]pyridazine in 400 ml of a mixture of chloroform and methanol (9/1). After reaction for 2 hours, a further 28.9 ml (28.9 mmol) of a 1M solution of iodine chloride in dichloromethane are added and the reaction is continued for 1 hour. The solution is then poured into a saturated solution of potassium bicarbonate and the mixture is decoloured by adding a 5% aqueous solution of sodium thiosulphate. The organic phase is separated, dried over sodium sulphate and concentrated under reduced pressure, to give a yellowish solid which is purified by silica gel column chromatography, elution being carried out with dichloromethane, to give 5.9 g of a mixture of 6-chloro-3-iodo-2-(5-chlorothien-2-yl)-imidazo[1,2-b]pyridazine and 6-chloro-3-chloro-2-(5-chlorothien-2-yl)imidazo[1,2-b]pyridazine (approximately 4/6) in the form of a yellow solid after trituration in 100 ml of acetonitrile, filtration and drying.

M+H=395 and 303

¹H NMR (DMSOd₆) δ: 8.30 and 8.20 (d and d, 1H); 7.85 and 7.65 (d and d, 1H); 7.48 and 7.54 (d and d, 1H); 7.26 and 7.28 (d and d, 1H) ppm.

### Stage 5.3. 6-Chloro-2-(5-chlorothien-2-yl)-3-(pyridin-4-yl)imidazo[1,2-b]pyridazine

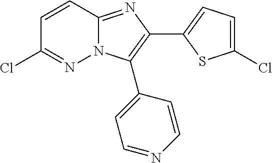 [SMILES: Clc1ccc2nc(-c3ccc(Cl)s3)c(-c3ccncc3)n2n1]

5.0 g (15 mmol) of caesium carbonate and 0.37 g (0.46 mmol) of a complex of [1,1″-bis(diphenylphosphino)ferrocene]dichloropalladium(II) and dichloromethane (PdCl₂(dppf).CH₂Cl₂) are added, after degassing using argon, to a mixture of 5.05 g (estimated at 5 mmol) of 6-chloro-3-iodo-2-(5-chlorothien-2-yl)imidazo[1,2-b]pyridazine and 6-chloro-3-chloro-2-(5-chlorothien-2-yl)imidazo[1,2-b]pyridazine (approximately 4/6) obtained in the previous stage and 1.26 g (6.12 mmol) of 4-(4,4,5,5-tetramethyl-1,3,2-dioxoborolan-2-yl)pyridine in 150 ml of a mixture of tetrahydrofuran and water (90/10). The reaction is stirred at reflux for 18 hours. The mixture is poured into a 1N aqueous solution of hydrochloric acid and the aqueous phase is washed with ethyl acetate. The aqueous phase is then basified using aqueous ammonia and the product is extracted with dichloromethane. The organic phase is dried over sodium sulphate and the solvent is evaporated off under reduced pressure. The residue is purified by chromatography on a 110 g silica gel column, elution being carried out with a mixture of dichloromethane, methanol and aqueous ammonia (98/2/0.2), to give 0.80 g of 6-chloro-2-(5-chlorothien-2-yl)-3-(pyridin-4-yl)imidazo[1,2-b]pyridazine in the form of a yellow solid.

¹H NMR (CDCl₃) δ: 8.80 (d, 2H); 8.30 (d, 1H); 7.70 (d, 2H); 7.50 (d, 1H); 7.10 (d, 1H); 7.00 (d, 1H) ppm.

### Stage 5.4. 2-{4-[2-(5-Chlorothien-2-yl)-3-(pyridin-4-yl)imidazo[1,2-b]pyridazin-6-yl](piperazin-1-yl)}ethanol

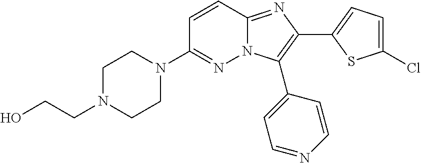 [SMILES: OCCN1CCN(c2ccc3nc(-c4ccc(Cl)s4)c(-c4ccncc4)n3n2)CC1]

A mixture of 0.20 g (0.58 mmol) of 6-chloro-2-(5-chlorothien-2-yl)-3-(pyridin-4-yl)imidazo[1,2-b]pyridazine and 0.65 g (2.9 mmol) of 2-(piperazin-1-yl)ethanol (CAS 103-76-4) in 3 ml of pentanol is refluxed for 24 hours at 145° C. The reaction medium is cooled and 5 ml of 3N aqueous hydrochloric acid (15 mmol) are added. The mixture is stirred for one hour and then diluted with water. The aqueous phase is washed with diethyl ether and then basified with 2N sodium hydroxide, and the product is extracted with dichloromethane. The organic phase is dried over sodium sulphate and the solvent is evaporated off under reduced pressure. The residue is purified by chromatography on a 50 g silica gel column, elution being carried out with a mixture of dichloromethane, methanol and aqueous ammonia (93/7/0.7), to give 0.17 g of 2-{4-[2-(5-chlorothien-2-yl)-3-(pyridin-4-yl)imidazo[1,2-b]pyridazin-6-yl](piperazin-1-yl)}ethanol in the form of a beige solid after crystallization from 20 ml of acetonitrile, filtration and drying.

Mp: 216-218° C.

¹H NMR (CDCl₃) δ: 8.65 (d, 2H); 7.70 (d, 1H); 7.60 (d, 2H); 6.90 (d, 1H); 6.85 (d, 1H); 6.70 (d, 1H); 3.6 (m, 2H); 3.40 (m, 4H); 2.55 (m, 7H) ppm.

### EXAMPLE NO. 6

### (Compound No. 20): 6-(Hexahydropyrrolo[3,4-c]pyrrol-2-yl)-3-(pyridin-4-yl)-2-(thien-3-yl)imidazo[1,2-b]pyridazine

### Stage 6.1. 6-Chloro-2-(thien-3-yl)imidazo[1,2-b]pyridazine

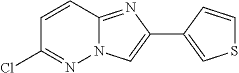 [SMILES: Clc1ccc2nc(-c3ccsc3)cn2n1]

A solution of 5.30 g (40.9 mmol) of 3-amino-6-chloropyridazine and 10 g (49 mmol) of 2-bromo-1-(thien-3-yl)ethanone (CAS 1468-82-2), added portionwise to 250 ml of ethanol, is refluxed for 2 hours. After cooling, the solvent is evaporated under reduced pressure, the orangey solid residue is taken up with chloroform and the solution is neutralized with an aqueous ammonia solution. The organic phase is separated and dried over sodium sulphate, to give 12 g of an orangey-brown solid after evaporation of the solvent. The solid is triturated in 100 ml of diisopropyl ether and isopropanol, to give 5.2 g of 6-chloro-2-(thien-3-yl)imidazo[1,2-b]pyridazine in the form of a orangey-beige solid after filtration and drying under reduced pressure.

Mp 203-205° C.

¹H NMR ¹H (DMSOd₆) δ: 8.80 (s, 1H); 8.20 (d, 1H); 8.05 (t, 1H); 7.50 (m, 2H); 7.40 (d, 1H) ppm.

### Stage 6.2. 6-Chloro-3-iodo-2-(thien-3-yl)imidazo[1,2-b]pyridazine

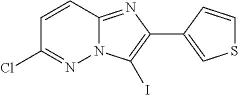 [SMILES: Clc1ccc2nc(-c3ccsc3)c(I)n2n1]

21.9 ml (21.9 mmol) of a 1M solution of iodine chloride in dichloromethane are added, at ambient temperature, to a solution of 3.69 g (15.6 mmol) of 6-chloro-2-(thien-3-yl)-imidazo[1,2-b]pyridazine in 170 ml of a mixture of chloroform and methanol (9/1). After reaction for 1 and a half hours, 100 ml of chloroform and a further 21.9 ml (21.9 mmol) of a 1M solution of iodine chloride in dichloromethane are added and the reaction is continued for 1 hour. The solution is then poured into a saturated solution of sodium bicarbonate and the mixture is decoloured by adding a 5% aqueous solution of sodium thiosulphate. The organic phase is separated, dried over sodium sulphate and concentrated under reduced pressure, to give an orangey solid which is purified by trituration in 50 ml of acetonitrile, filtration and drying, so as to give 4.9 g of 6-chloro-3-iodo-2-(thien-3-yl)imidazo[1,2-b]pyridazine in the form of a yellow solid after trituration in 50 ml of acetonitrile, filtration and drying.

Mp: 203-206° C.

¹H NMR (DMSOd₆) δ: 8.30 (dd, 1H); 8.15 (d, 1H); 7.90 (dd, 1H); 7.75 (dd, 1H); 7.50 (d, 1H) ppm.

### Stage 6.3. 6-Chloro-3-(pyridin-4-yl)-2-(thien-3-yl)imidazo[1,2-b]pyridazine

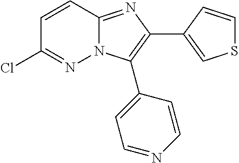 [SMILES: Clc1ccc2nc(-c3ccsc3)c(-c3ccncc3)n2n1]

9.0 g (28 mmol) of caesium carbonate and 0.68 g (0.83 mmol) of a complex of [1,1′-bis(diphenylphosphino)ferrocene]dichloropalladium(II) and dichloromethane (PdCl₂(dppf).CH₂Cl₂) are added, after degassing using argon, to a mixture of 3.35 g (9.26 mmol) of 6-chloro-3-iodo-2-(thien-3-yl)imidazo[1,2-b]pyridazine and 2.28 g (11.1 mmol) of 4-(4,4,5,5-tetramethyl-1,3,2-dioxoborolan-2-yl)pyridine (CAS 181219-01-2) in 120 ml of a mixture of tetrahydrofuran and water (9/1). The mixture is stirred at reflux for 18 hours and is then poured into 350 ml of a 1N aqueous solution of hydrochloric acid and the aqueous phase is washed with ethyl acetate. The aqueous phase is then basified using aqueous ammonia and the product is extracted with chloroform. The organic phase is dried over sodium sulphate and the solvent is evaporated off under reduced pressure. The residue is purified by chromatography on a 90 g silica gel column, elution being carried out with a mixture of dichloromethane, methanol and aqueous ammonia (97/3/0.3), to give 1.75 g of 6-chloro-3-(pyridin-4-yl)-2-(thien-3-yl)imidazo[1,2-b]pyridazine in the form of a yellow solid after trituration in diisopropyl ether, filtration and drying.

Mp: 225-231° C.

¹H NMR (DMSOd₆) δ: 8.80 (d, 2H); 8.30 (d, 1H); 7.75 (d, 1H); 7.65 (m, 3H); 7.50 (d, 1H); 7.25 (d, 1H) ppm.

### Stage 6.4. 6-(Hexahydropyrrolo[3,4-c]pyrrol-2(1H)-yl)-3-(pyridin-4-yl)-2-(thien-3-yl)imidazo[1,2-b]pyridazine

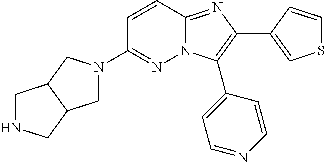 [SMILES: c1cc(-c2c(-c3ccsc3)nc3ccc(N4CC5CNCC5C4)nn23)ccn1]

A mixture of 0.350 g (1.12 mmol) of 6-chloro-3-(pyridin-4-yl)-2-(thien-3-yl)imidazo-[1,2-b]pyridazine and 0.475 g (2.24 mmol) of tert-butyl hexahydropyrrolo[3,4-c]pyrrole-2(1H)-carboxylate (CAS 141449-85-6) in 5 ml of pentanol is refluxed for 24 hours at 150° C. The reaction medium is cooled and approximately 5 ml of 3N aqueous hydrochloric acid (15 mmol) are then added. The mixture is stirred for one hour and then diluted with water. The aqueous phase is washed with ethyl acetate and then basified using aqueous ammonia, and the product is extracted with dichloromethane. The organic phase is dried over sodium sulphate and the solvent is evaporated off under reduced pressure. The brown oil obtained is purified by chromatography on a 35 g silica gel column, elution being carried out with a mixture of dichloromethane, methanol and aqueous ammonia (90/10/1), to give 0.235 g of 6-(hexahydropyrrolo[3,4-c]pyrrol-2(1H)-yl)-3-(pyridin-4-yl)-2-thien-3-ylimidazo[1,2-b]-pyridazine in the form of a beige solid after crystallization from 15 ml of acetonitrile, filtration and drying.

Mp: 196-198° C.

¹H NMR (CDCl₃) δ: 8.70 (d, 2H); 7.80 (d, 1H); 7.70 (d, 2H); 7.55 (d, 1H); 7.3 (m, 2H); 6.75 (d, 1H); 3.70 (m, 2H); 3.40 (dd, 2H); 3.20 (dd, 2H); 3.00 (m, 2H); 2.90 (dd, 2H) ppm.

### EXAMPLE NO. 7

### (Compound No. 32): 2-(Furan-2-yl)-6-[(cis)-5-methylhexahydro-pyrrolo[3,4-c]pyrrol-2(1H)-yl]-3-(pyridin-4-yl)imidazo[1,2-b]pyridazine

### Stage 7.1. 6-Chloro-2-(furan-2-yl)-3-iodoimidazo[1,2-b]pyridazine

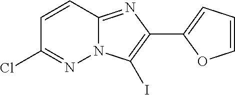 [SMILES: Clc1ccc2nc(-c3ccco3)c(I)n2n1]

3.39 g (30.0 mmol) of N-iodosuccinimide are added to a solution, at 60° C., of 5.49 g (25.0 mmol) of 6-chloro-2-(furan-2-yl)imidazo[1,2-b]pyridazine (J. Heterocyclic Chem., 2002, 39, 4, 737) in 200 ml of acetonitrile. After stirring for 2 hours, a further 1.41 g (12.5 mmol) of N-iodosuccinimide are added and the heating and also the stirring are continued for a further 2 hours. The solvent is then removed by evaporation under reduced pressure and the residue is taken up in a 1N solution of aqueous sodium hydroxide. Dichloromethane is then added and the mixture is treated, with vigorous stirring, with sodium thiosulphate, added portionwise until decolouration is obtained (red to pale yellow). The organic phase is separated, dried over sodium sulphate and concentrated under reduced pressure, to give a yellow solid which is purified by two successive rounds of chromatography on columns of 150 g and 120 g of silica gel, elution being carried out with dichloromethane and with a mixture of dichloromethane, methanol and aqueous ammonia (98/2/0.2), to give 1.9 g of 6-chloro-2-(furan-2-yl)-3-iodoimidazo[1,2-b]pyridazine containing 12% 6-chloro-2-(5-iodofuran-2-yl)-3-iodoimidazo[1,2-b]pyridazine, in the form of a solid.

Mp 260-263° C.

¹H NMR (CDCl₃) δ: 7.90 (d, 1H); 7.65 (s, 1H); 7.30 (dd, 1H); 7.20 (d, 1H); 6.65 (d, 1H) ppm.

### Stage 7.2. 6-Chloro-3-(pyridin-4-yl)-2-(furan-2-yl)imidazo[1,2-b]pyridazine

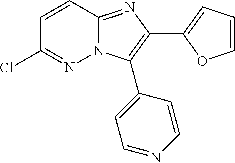 [SMILES: Clc1ccc2nc(-c3ccco3)c(-c3ccncc3)n2n1]

4.7 g (15 mmol) of caesium carbonate and 0.36 g (0.44 mmol) of a complex of [1,1′-bis(diphenylphosphino)ferrocene]dichloropalladium(II) and dichloromethane (PdCl₂(dppf).CH₂Cl₂) are added, after degassing using argon, to a mixture of 1.90 g (4.84 mmol) of 6-chloro-2-(furan-2-yl)-3-iodoimidazo[1,2-b]pyridazine and 1.29 g (6.29 mmol) of 4-(4,4,5,5-tetramethyl-1,3,2-dioxoborolan-2-yl)pyridine in 40 ml of a mixture of tetrahydrofuran and water (9/1). The reaction is stirred at reflux for 25 hours. The mixture is poured into 100 ml of a 1N aqueous solution of hydrochloric acid and the aqueous phase is washed with ethyl acetate. The aqueous phase is then basified using aqueous ammonia and the product is extracted with chloroform. The organic phase is dried over sodium sulphate and the solvent is evaporated off under reduced pressure. The solid brown residue is purified by chromatography on a 40 g silica gel column, elution being carried out with a mixture of dichloromethane, methanol and aqueous ammonia (98/2/0.2), to give 0.67 g of 6-chloro-3-(pyridin-4-yl)-2-(furan-2-yl)imidazo[1,2-b]pyridazine in the form of a cottonwool-like yellow solid after recrystallization from acetonitrile, filtration and drying.

Mp: 213-215° C.

¹H NMR (CDCl₃) δ: 8.85 (d, 2H); 8.00 (d, 1H); 7.70 (d, 2H); 7.50 (d, 1H); 7.20 (d, 1H); 6.85 (d, 1H); 6.55 (d, 1H) ppm.

### Stage 7.3. 2-(Furan-2-yl)-6-[(cis)-5-methylhexahydropyrrolo[3,4-c]pyrrol-2(1H)-yl]-3-(pyridin-4-yl)imidazo[1,2-b]pyridazine

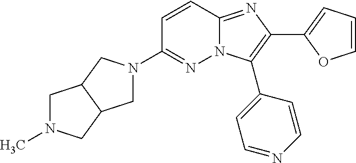 [SMILES: CN1CC2CN(c3ccc4nc(-c5ccco5)c(-c5ccncc5)n4n3)CC2C1]

A mixture of 0.300 g (0.10 mmol) of 6-chloro-3-(pyridin-4-yl)-2-(furan-2-yl)imidazo-[1,2-b]pyridazine, 0.255 g (2.02 mmol) of (cis)-octahydro-2-methylpyrrolo[3,4-c]pyrrole (CAS 172739-03-6) and 0.14 ml (1.01 mmol) of diisopropylethylamine in 5 ml of pentanol is refluxed for 18 hours at 150° C. The reaction medium is then cooled. The mixture is poured into 60 ml of a 1N aqueous solution of hydrochloric acid and the aqueous phase is washed with ethyl acetate. The aqueous phase is then basified using aqueous ammonia and the product is extracted with chloroform. The organic phase is dried over sodium sulphate and the solvent is evaporated off under reduced pressure. The residue is purified by chromatography on a 40 g silica gel column, elution being carried out with a mixture of dichloromethane, methanol and aqueous ammonia (90/10/1), to give 0.28 g of 2-(furan-2-yl)-6-[(cis)-5-methyl hexahydropyrrolo[3,4-c]pyrrol-2(1H)-yl]-3-(pyridin-4-yl)imidazo[1,2-b]pyridazine in the form of a beige powder after recrystallization from acetonitrile, filtration and drying.

Mp: 162-164° C.

¹H NMR (CDCl₃) δ: 8.75 (d, 2H); 7.80 (m, 3H); 7.50 (d, 1H); 6.75 (m, 2H); 6.50 (d, 1H); 3.7 (m, 2H); 3.4 (dd, 2H); 3.05 (m, 2H); 2.65 (m, 4H); 2.40 (s, 3H) ppm.

### EXAMPLE NO. 8

### (Compound No. 25): 2-(2,5-Dimethylthien-3-yl)-3-(2-methylpyridin-4-yl)-6-(piperazin-1-yl)imidazo[1,2-b]pyridazine

### Stage 8.1. 4-[2-(2,5-Dimethylthien-3-yl)imidazol-[1,2-b]pyridazin-6-yl]piperazine-1-carbaldehyde

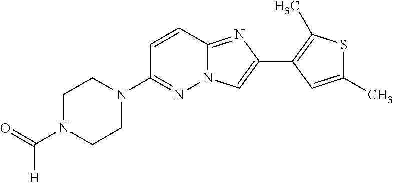 [SMILES: Cc1cc(-c2cn3nc(N4CCN(C=O)CC4)ccc3n2)c(C)s1]

A mixture of 3.02 g (16 mmol) of 1-(2,5-dimethylthien-3-yl)-2-bromoethanone, 4.47 g (21.5 mmol) of 4-(6-aminopyridazin-3-yl)piperazine-1-carbaldehyde and 1.5 g (15 mmol) of triethylamine in 10 ml of tert-butanol is heated in a microwave reactor at 140° C. for 30 minutes. The mixture is then diluted with water and the product is extracted with ethyl acetate. The organic phase is then washed with a saturated solution of sodium chloride and dried over sodium sulphate, and the solvent is evaporated off under reduced pressure with 8 g of silica gel. The product is then purified by chromatography on an 80 g silica gel column, elution being carried out with a gradient of 0 to 10% of methanol in dichloromethane, to give 1.81 g of 4-[2-(2,5-dimethylthien-3-yl)imidazo[1,2-b]pyridazin-6-yl]piperazine-1-carbaldehyde in the form of a slightly yellow solid.

¹H NMR (CDCl₃) δ: 8.18 (s, 1H); 7.8 (s, 1H); 7.79 (d, 1H); 7.16 (s, 1H); 6.8 (d, 1H); 3.4-3.8 (m, 8H); 2.62 (s, 3H); 2.4 (s, 3H).

### Stage 8.2. 4-[2-(2,5-Dimethylthien-3-yl)-3-iodoimidazo[1,2-b]pyridazin-6-yl]piperazine-1-carbaldehyde

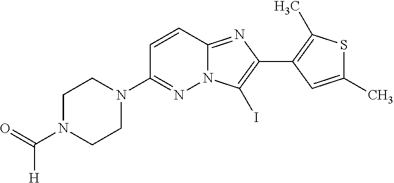 [SMILES: Cc1cc(-c2nc3ccc(N4CCN(C=O)CC4)nn3c2I)c(C)s1]

2.7 g (12 mmol) of N-iodosuccinimide are added portionwise to a solution of 3.4 g (10 mmol) of 4-[2-(2,5-dimethylthien-3-yl)imidazo[1,2-b]pyridazin-6-yl]piperazine-1-carbaldehyde in 80 ml of chloroform. The mixture is stirred at ambient temperature for two hours and then the mixture is diluted with dichloromethane and the solution is washed with an aqueous solution of sodium thiosulphite and with a saturated solution of sodium chloride. After drying over sodium sulphate and addition of silica gel, the solvent is evaporated under reduced pressure. The product is purified by chromatography on an 80 g silica gel column, elution being carried out with a gradient of 0 to 10% of methanol in dichloromethane, to give 3.35 g of 4-[2-(2,5-dimethylthien-3-yl)-3-iodoimidazo[1,2-b]pyridazin-6-yl]piperazine-1-carbaldehyde.

¹H NMR (CDCl₃) δ: 8.2 (s, 1H); 7.46 (d, 1H); 6.95 (s, 1H); 6.82 (d, 1H); 3.47-3.8 (m, 8H); 2.5 (s, 3H); 2.42 (5, 3H).

### Stage 8.3. 4-[2-(2,5-Dimethylthien-3-yl)-3-(2-methylpyridin-4-yl)imidazo[1,2-b]pyridazin-6-yl]piperazine-1-carbaldehyde

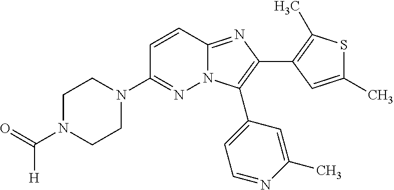 [SMILES: Cc1cc(-c2c(-c3cc(C)sc3C)nc3ccc(N4CCN(C=O)CC4)nn23)ccn1]

A mixture of 0.398 g (0.85 mmol) of 4-[2-(2,5-dimethylthien-3-yl)-3-iodoimidazo[1,2-b]pyridazin-6-yl]piperazine-1-carbaldehyde, 7.5 mg of [bis(diphenylphosphino)ferrocene]dichloropalladium(II) (Pd(dppf)₂Cl₂), 0.132 g (1 mmol) of 2-methylpyridine-4-boronic acid and 3 ml of a 2M aqueous solution of caesium carbonate in 12 ml of 1,4-dioxane is heated in a microwave reactor at 115° C. for 20 minutes. The mixture is then partitioned between 5 ml of a saturated aqueous solution of sodium chloride and 40 ml of ethyl acetate. The organic phase is dried over sodium sulphate and the solvent is evaporated off under reduced pressure with 1.5 g of silica gel. The product is then purified by chromatography on a 10 g silica gel column, elution being carried out with a gradient of 0 to 10% of methanol in dichloromethane, to give 0.295 g of 4-[2-(2,5-dimethylthien-3-yl)-3-(2-methylpyridin-4-yl)imidazo[1,2-b]pyridazin-6-yl]piperazine-1-carbaldehyde.

¹H NMR (CDCl₃) δ: 8.5 (d, 1H); 8.15 (s, 1H); 7.82 (d, 1H); 7.5 (s, 1H); 7.0 (d, 1H); 6.92 (d, 1H); 6.64 (s, 1H); 3.73 (m, 2H); 3.57 (m, 6H); 2.57 (s, 3H); 2.4 (s, 3H); 2.13 (s, 3H).

### Stage 8.4. 2-(2,5-Dimethylthien-3-yl)-3-(2-methylpyridin-4-yl)-6-piperazin-1-ylimidazo[1,2-b]pyridazine

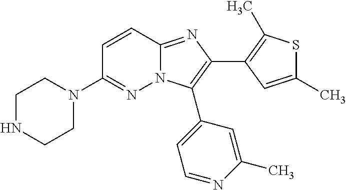 [SMILES: Cc1cc(-c2c(-c3cc(C)sc3C)nc3ccc(N4CCNCC4)nn23)ccn1]

A solution of 0.255 g (0.59 mmol) of 4-[2-(2,5-dimethylthien-3-yl)-3-(2-methylpyridin-4-yl)imidazo[1,2-b]pyridazin-6-yl]piperazine-1-carbaldehyde in 3.5 ml of tetrahydrofuran and 1 ml of sulphuric acid is heated at 105° C. for 10 minutes in a microwave reactor. The medium is basified by adding aqueous ammonia and the product is extracted with ethyl acetate. The organic phase is then dried over sodium sulphate and the solvent is evaporated off under reduced pressure with 1 g of silica gel. The product is then purified by chromatography on a 4 g silica gel column, elution being carried out with a gradient of 0 to 10% of methanol and 1% of aqueous ammonia in dichloromethane, to give 0.195 g of 2-(2,5-dimethylthien-3-yl)-3-(2-methylpyridin-4-yl)-6-piperazin-1-ylimidazo[1,2-b]pyridazine.

¹H NMR (CDCl₃) δ: 8.5 (d, 1H); 7.77 (d, 1H); 7.58 (s, 1H); 7.2 (d, 1H); 6.9 (d, 1H); 6.66 (s, 1H); 3.45 (m, 4H); 3.0 (m, 4H); 2.5 (s, 3H); 2.4 (s, 3H); 2.1 (s, 3H).

### EXAMPLE NO. 9

### (Compound No. 33): 2-(5-Methylfuran-2-yl)-6-[(cis)-5-methylhexahydropyrrolo[3,4-c]pyrrol-2(1H)-yl]-3-(pyridin-4-yl)imidazo[1,2-b]pyridazine

### Stage 9.1. 2-Bromo-6-[(cis)-5-methylhexahydropyrrolo[3,4-c]pyrrol-2-(1H)-yl]imidazo[1,2-b]pyridazine

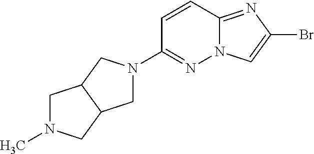 [SMILES: CN1CC2CN(c3ccc4nc(Br)cn4n3)CC2C1]

A mixture of 2.50 g (10.8 mmol) of 2-bromo-6-chloroimidazo[1,2-b]pyridazine (CAS 944902-75-4), 1.9 g (15 mmol) of (cis)-octahydro-2-methylpyrrolo[3,4-c]pyrrole (CAS 172739-03-6) and 1.5 ml (10.8 mmol) of diisopropylethylamine in 20 ml of pentanol is refluxed for 3 days at 150° C. The reaction medium is then cooled. The mixture is poured into 20 ml of a 1N aqueous solution of hydrochloric acid, and the aqueous phase is washed with ethyl acetate. The aqueous phase is then basified by means of 2M sodium hydroxide and the product is extracted with dichloromethane. The organic phase is dried over sodium sulphate and the solvent is evaporated off under reduced pressure. The residue is purified by chromatography on an 80 g silica gel column, elution being carried out with a mixture of dichloromethane, methanol and aqueous ammonia (93/7/0.7), to give 2.6 g of 2-bromo-6-[(cis)-5-methylhexahydropyrrolo[3,4-c]pyrrol-2(1H)-yl]imidazo[1,2-b]pyridazine in the form of a pale yellow solid after trituration from diisopropyl ether, filtration and drying.

Mp: 144-146° C.

¹H NMR (DMSO d₆) δ: 8.05 (s, 1H); 7.80 (d, 1H); 6.95 (d, 2H); 3.65 (dd, 2H); 3.30 (dd, 2H); 2.95 (m, 2H); 2.5 (m, 4H); 2.25 (s, 3H) ppm.

### Stage 9.2. 2-Bromo-3-iodo-6-[(cis)-5-methylhexahydropyrrolo[3,4-c]pyrrol-2(1H)-yl]imidazo[1,2-b]pyridazine

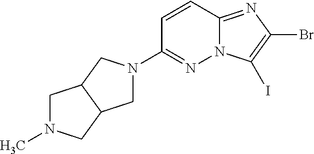 [SMILES: CN1CC2CN(c3ccc4nc(Br)c(I)n4n3)CC2C1]

18.8 g (18.8 mmol) of a 1M solution of iodine chloride in dichloromethane are added to a solution of 2.42 g (7.51 mmol) of 2-bromo-6-[(cis)-5-nnethylhexahydropyrrolo[3,4-c]pyrrol-2(1H)-yl]imidazo[1,2-b]pyridazine in 150 ml of a mixture of dichloromethane and methanol (8/2). After stirring for one and a half hours, a saturated aqueous solution of sodium bicarbonate and then an aqueous sodium thiosulphate solution at 5% are successively added until discoloration occurs. The organic phase is separated, dried over sodium sulphate and concentrated under reduced pressure, so as to give a brown solid which is triturated with 15 ml of acetonitrile, to give 2.65 g of 2-bromo-3-iodo-6-[(cis)-5-methylhexahydropyrrolo[3,4-c]pyrrol-2(1H)-yl]imidazo[1,2-b]pyridazine, in the form of a whitish powder.

Mp: 208-212° C.

¹H NMR (DMSO d₆) δ: 7.75 (d, 1H), 6.95 (d, 1H); 3.70 (dd, 2H); 3.40 (dd, 2H); 2.95

### Stage 9.3. 2-Bromo-6-[(cis)-5-methylhexahydropyrrolo[3,4-c]pyrrol-2(1H)-yl]-3-pyridin-4-yl)imidazo[1,2-b]pyridazine

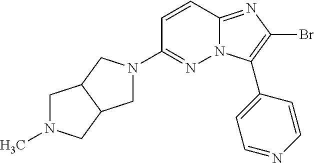 [SMILES: CN1CC2CN(c3ccc4nc(Br)c(-c5ccncc5)n4n3)CC2C1]

0.43 g (0.53 mmol) of a complex of 1,1′-bis(diphenylphosphino)ferrocenedichloropalladium (II) and of dichloromethane (PdCl₂(dppf).CH₂Cl₂—CAS 851232-71-8) is added, after degassing with argon, to a mixture of 2.65 g (5.91 mmol) of 2-bromo-3-iodo-6-[(cis)-5-methylhexahydropyrrolo[3,4-c]pyrrol-2(1H)-yl]imidazo[1,2-b]pyridazine, 6.51 g (6.29 mmol) of 4-(4,4,5,5-tetramethyl-1,3,2-dioxoborolan-2-yl)pyridine (CAS 181219-01-2), and 5.7 g (18 mmol) of cesium carbonate in 120 ml of a mixture of tetrahydrofuran and water (9/1). The reaction is stirred at reflux for 24 hours. The mixture is poured into a 1N aqueous solution of hydrochloric acid, and the aqueous phase is washed with ethyl acetate. The aqueous phase is then basified by means of aqueous ammonia and the product is extracted with dichloromethane. The organic phase is dried over sodium sulphate and the solvent is evaporated off under reduced pressure. The solid brown residue is purified by chromatography on a 150 g silica gel column, elution being carried out with a mixture of dichloromethane, methanol and aqueous ammonia (98/2/0.2), to give 1.26 g of 2-bromo-6-[(cis)-5-methylhexahydropyrrolo[3,4-c]pyrrol-2-(1H)-yl]-3-pyridin-4-yl)imidazo[1,2-b]pyridazine in the form of a beige powder after crystallization from diisopropyl ether, filtration and drying.

Mp: 195-197° C.

¹H NMR (DMSO d₆) δ: 8.75 (d, 2H); 8.00 (d, 2H); 7.90 (d, 1H); 7.10 (d, 1H); 3.65 (dd, 2H); 3.35 (dd, 2H); 2.95 (d, 2H); 2.5 (m, 4H); 2.20 (s, 3H) ppm.

### Stage 9.4. 2-(5-Methylfuran-2-yl)-6-[(cis)-5-methylhexahydropyrrolo[3,4-c]pyrrol-2(1H)-yl]-3-(pyridin-4-yl) imidazo[1,2-b]pyridazine

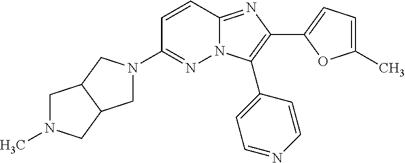

0.076 g (0.09 mmol) of a complex of 1,1′-bis(diphenylphosphino)ferrocenedichloropalladium (II) and of dichloromethane (PdCl₂(dppf).CH₂Cl₂) is added, after degassing with argon, to a mixture of 0.410 g (1.03 mmol) of 2-bromo-6-[(cis)-5-methylhexahydropyrrolo[3,4-c]pyrrol-2(1H)-yl]-3-(pyridin-4-yl)imidazo[1,2-b]pyridazine, 1.00 g (3.08 mmol) of cesium carbonate and 0.162 g (1.28 mmol) of 5-methylfuran-2-boronic acid (CAS 62306-79-0) in 40 ml of a mixture of tetrahydrofuran and water (9/1). The reaction is stirred at reflux for 24 hours. The mixture is poured into 100 ml of a 1N aqueous solution of hydrochloric acid, and the aqueous phase is washed with ethyl acetate. The aqueous phase is then basified by means of a 2N aqueous solution of sodium hydroxide and the product is extracted with dichloromethane. The organic phase is dried over sodium sulphate and the solvent is evaporated off under reduced pressure. The solid brown residue is purified by chromatography on a 40 g silica gel column, elution being carried out with a mixture of dichloromethane, methanol and aqueous ammonia (94/6/0.6), to give 0.35 g of 2-(5-methylfuran-2-yl)-6-[(cis)-5-methylhexahydropyrrolo[3,4-c]pyrrol-2(1H)-yl]-3-(pyridin-4-yl)imidazo[1,2-b]pyridazine in the form of a beige solid after recrystallization from 8 ml of acetonitrile, filtration and drying.

Mp: 178-181° C.

¹H NMR (CDCl₃) δ: 8.75 (d, 2H); 7.8 (m, 3H); 670 (d, 2H); 6.55 (d, 1H); 6.05 (d, 1H); 3.65 (dd, 2H); 3.40 (dd, 2H); 3.00 (m, 2H); 270 (m, 2H); 2.60 (m, 2H); 2.35 (s and S. 3H and 3H) ppm.

Table 1 which follows illustrates the chemical structures and the physical properties of some compounds according to the invention.

In this table:
the “Mp° C.” column gives the melting points of the products in degrees Celsius. “N.D.” means that the melting point is not determined,
in the “Salt” column, “HCl” represents a compound in hydrochloride form and the ratio between parentheses is the (acid:base) ratio, the sign “-” means that the compound is in the form of a base,
the “m/z” column gives the molecular ion (M+H⁺) observed by analysis of the products by mass spectrometry, either by LC-MS (liquid chromatography coupled to Mass Spectroscopy) carried out on an Agilent LC-MSD Trap apparatus in the positive ESI mode, or by direct introduction by MS (Mass Spectroscopy) on an Autospec M (EBE) apparatus using the DCI—NH₃ technique or using the electron impact technique on a Waters GCT apparatus.-
“CH₃—” means methyl,
“NH₂—” means amino,
“CH₃OH” means methanol,
“DMSO” means dimethyl sulphoxide.

TABLE 1

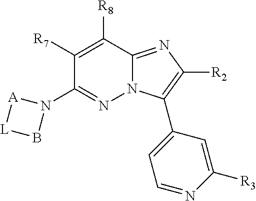

| N^{o} | —N—A—L—B— | R₇ | R₈ | R₂ | R₃ | Salt | Mp ° C. | M + H |
|---|---|---|---|---|---|---|---|---|
| 1 | Piperazin-1-yl | H | H | thien-2-yl | H | — | 217-220 | 363 |
| 2 | Piperazin-1-yl | H | H | thien-2-yl | CH₃ | — | N.D. | 377 |
| 3 | 3-Methylpiperazin-1-yl | H | H | thien-2-yl | H | — | N.D. | 377 |
| 4 | 4-(2-hydroxyethyl)piperazin- | H | H | thien-2-yl | H | — | N.D. | 407 |
| | 1-yl | | | | | | | |
| 5 | 4-(2-Hydroxy-2-methylpropyl) | H | H | thien-2-yl | H | — | 165-168 | 435 |
| | piperazin-1-yl | | | | | | | |
| 6 | (cis)-Hexahydropyrrolo[3,4-c] | H | H | thien-2-yl | H | — | 179-183 | 389 |
| | pyrrol-2(1H)-yl | | | | | | | |
| 7 | Octahydro-6H-pyrrolo[3,4-b] | H | H | thien-2-yl | H | — | 176-179 | 403 |
| | pyridin-6-yl | | | | | | | |
| 8 | 2,9-Diazaspiro[5.5]undec-9-yl | H | H | thien-2-yl | H | — | 184-189 | 431 |
| 9 | 4-Pyrrolidin-1-ylpiperidin-1-yl | H | H | thien-2-yl | H | — | 85 | 431 |
| | | | | | | | (Trans- | |
| | | | | | | | formation) | |
| 10 | Piperazin-1-yl | H | H | 5-methyl-thien-2-yl | H | — | N.D. | 377 |
| 11 | Piperazin-1-yl | H | H | 5-methyl-thien-2-yl | CH₃— | — | N.D. | 391 |
| 12 | Piperazin-1-yl | H | H | 5-methyl-thien-2-yl | NH₂— | — | N.D. | 392 |
| 13 | (cis)-3,5-dimethylpiperazin- | H | H | 5-chloro-thien-2-yl | H | — | 220-222 | 425 |
| | 1-yl | | | | | | | |
| 14 | 4-(2-hydroxyethyl)piperazin- | H | H | 5-chloro-thien-2-yl | H | — | 216-218 | 441 |
| | 1-yl | | | | | | | |
| 15 | (cis)-5-Methyl-hexahydro- | H | H | 5-chloro-thien-2-yl | H | — | 217-220 | 437 |
| | pyrrolo[3,4-c]pyrrol-2(1H)-yl | | | | | | | |
| 16 | Octahydro-6H-pyrrolo[3,4-b] | H | H | 5-chloro-thien-2-yl | H | — | 239-241 | 437 |
| | pyridin-6-yl | | | | | | | |
| 17 | Piperazin-1-y1 | H | H | thien-3-y1 | NH₂— | — | N.D. | 378 |
| 18 | 4-Methylpiperazin-1-yl | H | H | thien-3-yl | H | — | 177-179 | 377 |
| 19 | 4-(2-Hydroxy-2-methyl-propyl) | H | H | thien-3-yl | H | — | 178-180 | 435 |
| | piperazin-1-yl | | | | | | | |
| 20 | (cis)-Hexahydropyrrolo[3,4-c] | H | H | thien-3-yl | H | — | 196-198 | 398 |
| | pyrrol-2(1H)-yl | | | | | | | |
| 21 | Octahydro-6H-pyrrolo[3,4-b] | H | H | thien-3-yl | H | HCl(3:1) | N.D. | 403 |
| | pyridin-6-yl | | | | | | | |
| 22 | 2,9-Diazaspiro[5.5]undec-9-yl | H | H | thien-3-yl | H | — | 133-168 | 431 |
| 23 | 4-pyrrolidin-1-yl-piperidin-1-yl | H | H | thien-3-yl | H | — | 168-170 | 431 |
| 24 | Piperazin-1-y1 | H | H | 2,5-dimethyl-thien-3-yl | H | — | N.D. | 391 |
| 25 | Piperazin-1-yl | H | H | 2,5-dimethyl-thien-3-yl | CH₃— | — | N.D. | 405 |
| 26 | Piperazin-1-y1 | H | H | 2,5-dimethyl-thien-3-yl | NH₂— | — | N.D. | 406 |
| 27 | 3,3-Dimethylpiperazin-1-yl | H | H | 2,5-dichloro-thien-3-yl | H | — | N.D. | 459 |
| 28 | 4-(2-Hydroxyethyl)piperazin- | H | H | 5-methyl-furan-2-yl | H | — | 190-192 | 405 |
| | 1-yl | | | | | | | |
| 29 | 4-(2-Hydroxy-2-methylpropyl) | H | H | 5-methyl-furan-2-yl | H | | 147-149 | 433 |
| | piperazin-1-yl | | | | | | | |
| 30 | 4-(2-Hydroxyethyl)piperazin- | H | H | furan-3-y1 | H | | 187-189 | 391 |
| | 1-yl | | | | | | | |
| 31 | 4-(2-Hydroxy-2-methylpropyl) | H | H | furan-3-yl | H | | 139-149 | 419 |
| | piperazin-1-yl | | | | | | | |
| 32 | (cis)-5-Methylhexahydro- | H | H | furan-2-yl | H | — | 162-164 | 387 |
| | pyrrolo[3,4-c]pyrrol-2(1H)-yl | | | | | | | |
| 33 | (cis)-5-Methylhexahydro- | H | H | 5-methyl-furan-2-yl | H | | 178-181 | 401 |
| | pyrrolo[3,4-c]pyrrol-2(1H)-yl | | | | | | | |
| 34 | (cis)-5-Methylhexahydro- | H | H | furan-3-yl | H | H | 144-148 | 387 |
| | pyrrolo[3,4-c]pyrrol-2(1H)-yl | | | | | | | |

### Biological Examples

The capacity of the compounds of the invention to inhibit the phosphorylation of casein by casein kinase 1 epsilon and delta can be evaluated according to the procedure described in document US 2005/0131012.

### Filter-Plate Assay of ATP-³³P for the Screening of CK1 Epsilon Inhibitors:

The effect of the compounds on inhibition of the phosphorylation of casein by the enzyme casein kinase 1 epsilon (CK1 epsilon) is measured using a casein assay with filtration of ATP-³³P in vitro.

Casein kinase 1 epsilon (0.58 mg/ml) is obtained via fermentation and purification processes carried out according to methods well known to those skilled in the art, or may also be obtained from Invitrogen Corporation™ (human CK1 epsilon).

The compounds are tested at five different concentrations so as to generate IC₅₀ values, i.e. the concentration at which a compound is capable of inhibiting the enzymatic activity by 50%, or alternatively the % inhibition at a concentration of 10 micromolar.

“U”-bottomed Falcon plates are prepared by placing 5 μl of solutions of the compounds according to the invention at concentrations of 10, 1, 0.1, 0.01 or 0.001 μM in various wells. The solutions of the compounds according to the invention at these various concentrations are prepared by diluting in a test buffer (50 mM Tris, pH 7.5, 10 M MgCl₂, 2 mM DTT and 1 mM EGTA) a stock solution in DMSO at a concentration of 10 mM. Next, 5 μl of dephosphorylated casein are added to a final concentration of 0.2 μg/μl, 20 μl of CK1 epsilon are added to a final concentration of 3 ng/μl, and 20 μl of ATP-³³P are added to a final concentration of 0.02 μCi/μl mixed with cold ATP (10 μM final—approximately 2×10⁶ CPM per well). The final total test volume per well is equal to 50 μl.

The “U”-bottomed Falcon® test plate mentioned above is vortexed, and then incubated at ambient temperature for 2 hours. After 2 hours, the reaction is stopped by adding an ice-cold solution of 65 μl of cold ATP (2 mM) prepared in test buffer. 100 μl of the reaction mixture are then transferred from the “U”-bottomed Falcon®plate into Millipore MAPH filter plates, preimpregnated with 25 μl of ice-cold 100% TCA.

The Millipore MAPH filter plates are agitated gently and are left to stand at ambient temperature for at least 30 minutes in order to precipitate the proteins.

After 30 minutes, the filter plates are sequentially washed and filtered with 2×150 μl of 20% TCA, 2×150 μl of 10% TCA and 2×150 μl of 5% TCA (6 washes in total per plate/900 μl per well).

The plates are left to dry overnight at ambient temperature. Next, 40 μl of Microscint-20 Packard® scintillation fluid are added per well and the plates are closed in a leaktight manner. The radiation emitted by each well is then measured for 2 minutes in a Packard® Topcount NXT scintillation counter, in which the values of CPM/well are measured.

The % inhibition of the capacity of the enzyme to phosphorylate the substrate (casein) is determined for each concentration of compound tested. These inhibition data expressed as percentages are used to calculate the IC₅₀ value for each compound compared with the controls.

The kinetic studies determined the K_{M} value for ATP as being 21 μM in this test system.

Table 2 below gives the IC₅₀ values for the inhibition of phosphorylation by casein kinase 1 epsilon for a number of compounds according to the invention.

TABLE 2

| Compound | |
|---|---|
| No. | CK1 epsilon IC₅₀ (nM) |
| 10 | 87 |
| 14 | 19 |
| 18 | 25 |
| 34 | 15 |

Under these conditions, the most active compounds of the invention show IC₅₀ values (concentration which inhibits 50% of the enzymatic activity of casein kinase 1 epsilon) of between 1 nM and 2 μM.

The capacity of the compounds of the invention to inhibit the phosphorylation of casein by casein kinase 1 epsilon and casein kinase 1 delta can be evaluated using a FRET (Fluorescence Resonance Energy Transfer) fluorescence test by means of the “Z′Lyte™ kinase assay kit” (reference PV3670; Invitrogen Corporation™) according to the supplier's instructions.

The casein kinases 1 used are obtained from Invitrogen Corporation (human CK1 epsilon PV3500 and human CK1 delta PV3665).

A peptide substrate, labelled at both ends with a fluorophore donor group (coumarin) and a fluorophore acceptor group (fluorescein) constituting a FRET system is phosphorylated in the presence of ATP by casein kinase 1 epsilon or delta in the presence of increasing concentrations of compounds of the invention.

The mixture is treated with a site-specific protease that specifically cleaves the peptide substrate so as to form two fluorescent fragments having a large fluorescence emission ratio.

The fluorescence observed is thus related to the capacity of the products of the invention to inhibit the phosphorylation of the peptide substrate by casein kinase 1 epsilon or casein kinase 1 delta.

The compounds of the invention are dissolved at various concentrations starting from a 10 mM stock solution in DMSO diluted in a buffer containing 50 mM HEPS, pH 7.5, 1 mM EGTA, 0.01% Brij-35, 10 mM MgCl₂ for casein kinase 1 epsilon and supplemented with Trizma Base (50 mM), pH 8,0, and NaN₃ (0.01% final) for casein kinase 1 delta.

The phosphorylation of the peptide substrate SER/THR 11 obtained from Invitrogen Corporation™ is performed at a final concentration of 2 μM. The ATP concentration is 4 times the K_{M}, this value being 2 μM for casein kinase 1 epsilon and 4 μM for casein kinase 1 delta.

The emitted fluorescence is measured at wavelengths of 445 and 520 nm (excitation at 400 nm).

Table 3 below gives the IC₅₀ values for inhibition of phosphorylation by casein kinase 1 delta for a number of compounds according to the invention.

TABLE 3

| Compound | |
|---|---|
| No. | CK1 delta IC₅₀ (nM) |
| 10 | 63-76 |
| 14 | 93-163 |

Under these conditions, the compounds of the invention that are the most active have IC₅₀ values (concentration that inhibits 50% of the enzymatic activity of casein kinase 1 delta) of between 1 nM and 2 μM.

It thus appears that the compounds according to the invention have an inhibitory activity on the casein kinase 1 epsilon or casein kinase 1 delta enzyme.

### Experimental Protocols for Circadian Cell Assay

Mper1-luc Rat-1 (P2C4) fibroblast cultures were prepared by dividing the cultures every 3-4 days (approximately 10-20% of confluence) on 150 cm² degassed polystyrene tissue culture flasks (Falcon® #35-5001) and maintained in growth medium [EMEM (Cellgro #10-010-CV); 10% foetal bovine serum (FBS; Gibco #16000-044); and 50 I.U./ml of penicillin-streptomycin (Cellgro #30-001-CI)] at 37° C. and under 5% CO₂.

Cells obtained from Rat-1 fibroblast cultures at 30-50% of confluence as described above were co-transfected with vectors containing the selectable marker for zeocin resistance for a stable transfection and a luciferase reporter gene controlled by the mPer-1 promoter. After 24 to 48 hours, the cultures were divided on 96-well plates and maintained in growth medium supplemented with 50-100 μg/ml of zeocin (Invitrogen® #45-0430) for 10-14 days. The zeocin-resistant stable transfectants were evaluated for the expression of the reporter gene by adding 100 μM luciferin (Promega® #E1603®) to the growth medium and by assaying the luciferase activity on a TopCount® scintillation counter (Packard Model #C384V00). The Rat-1 cell clones expressing both zeocin resistance and luciferase activity controlled by mPer1 were serum-shock synchronized with 50% horse serum [HS (Gibco® #16050-122)] and the activity of the circadian reporter was evaluated. The P2C4 clone of Mper1-luc Rat-1 fibroblasts was selected to test the compound.

Mper1-luc Rat-1 (P2C4) fibroblasts at 40-50% of confluence, obtained according to the protocol described above, were plated out onto 96-well opaque tissue culture plates (Perkin Elmer® #6005680). The cultures are maintained in growth medium supplemented with 100 μg/mL of zeocin (Invitrogen #45-0430) until they have reached 100% of confluence (48-72 h). The cultures were then synchronized with 100 μl of synchronization medium [EMEM (Cellgro #10-010-CV); 100 I.U./ml of penicillin-streptomycin (Cellgro #30-001-C1); HS at 50% (Gibco #16050-122)] for 2 hours at 37° C. and under 5% CO₂. After synchronization, the cultures were rinsed with 100 μl of EMEM (Cellgro #10-010-CV) for 10 minutes at ambient temperature. After rinsing, the medium was replaced with 300 μl of CO₂ independent medium [CO₂I (Gibco #18045-088); 2 mM L-glutamine (Cellgro #25-005-C1); 100 I.U./ml of penicillin-streptomycin (Cellgro #30-001-C1); 100 μM luciferin (Promega #E 1603)]. The compounds of the invention tested for the circadian effects were added to CO₂-independent medium in DMSO at 0.3% (final concentration). The cultures were immediately closed in a leaktight manner with TopSeal-A® film (Packard #6005185) and transferred for the luciferase activity measurement.

After synchronization, the test plates were maintained at 37° C. in a tissue culture incubator (Form a Scientific Model #3914). The in vivo luciferase activity was estimated by measuring the relative light emission on a TopCount scintillation counter (Packard Model #C384V00).

The period analysis was performed either by determining the interval between the relative light emission minima over several days or by Fourier transform. The two methods produced a virtually identical period estimation over a range of circadian periods. The power is reported in CE Delta (t+1 h), which is presented as the effective micromolar concentration that induced a 1-hour prolongation of the period. The data were analysed by adjusting a hyperbolic curve to the data expressed as change of period (Y-axis) as a function of the concentration of the test compound (X-axis) in the XLfit™ software, and the CE Delta (t+1 h) was interpolated from this curve.

Table 4 below gives the CE Delta (t+1 h) for a number of compounds according to the invention.

TABLE 4

| Compound | |
|---|---|
| No. | CE Delta (t + 1 h) (nM) |
| 10 | 360 |
| 14 | 60-117 |
| 18 | 74-83 |
| 34 | 17 |

Under these conditions, the compounds of the invention that are the most active have CE Delta (t+1 h) (effective micromolar concentration that induced a 1-hour prologation of the period) of between 1 nM and 2 μM.

By inhibiting the CK1 epsilon and/or CK1delta enzymes, the compounds which are subjects of the invention modulate the circadian periodicity, and may be useful for the treatment of circadian rhythm-related disorders.

The compounds according to the invention may in particular be used for the preparation of a medicament for preventing or treating sleep disorders; circadian rhythm disorders, such as, in particular, those caused by jetlag or shift work.

Among the sleep disorders, especially distinguished are primary sleep disorders such as dyssomnia (for example, primary insomnia), parasomnia, hypersomnia (for example excessive drowsiness), narcolepsy, sleep disorders related to sleep apnoea, sleep disorders related to the circadian rhythm and otherwise unspecified dyssomnias, sleep disorders associated with medical/psychiatric disorders.

The compounds which are subjects of the invention also cause a circadian phase shift and such a property may be useful in the context of a potential monotherapy or combined therapy that is clinically effective in the case of mood disorders.

Among the mood disorders, especially distinguished are depressive disorders (unipolar depression), bipolar disorders, mood disorders caused by a general medical complaint and also mood disorders induced by pharmacological substances.

Among the bipolar disorders, especially distinguished are bipolar I disorders and bipolar II disorders, including in particular seasonal affective disorders.

The compounds which are subjects of the invention, which modulate circadian rhythm, may be useful in the treatment of anxiety and depressive disorders caused in particular by an impairment in the secretion of CRF.

Among the depressive disorders, especially distinguished are major depressive disorders, dysthymic disorders and otherwise unspecified depressive disorders.

The compounds which are subjects of the invention, which modulate circadian rhythm, may be useful for the preparation of a medicament for treating diseases related to dependence on abuse substances such as cocaine, morphine, nicotine, ethanol or cannabis.

By inhibiting casein kinase 1 epsilon and/or casein kinase 1 delta, the compounds according to the invention may be used for the preparation of medicaments, in particular for the preparation of a medicament for preventing or treating diseases related to hyperphosphorylation of the tau protein, in particular Alzheimer's disease.

These medicaments also find their use in therapy, in particular in the treatment or prevention of diseases caused or exacerbated by cell proliferation, in particular tumour cell proliferation.

As tumour cell proliferation inhibitors, these compounds are useful in the prevention and treatment of liquid tumours such as leukaemias, solid tumours that are both primary and metastatic, carcinomas and cancers, in particular: breast cancer; lung cancer; cancer of the small intestine, colorectal cancer; cancer of the respiratory pathways, of the oropharynx and of the hypopharynx; oesophageal cancer; liver cancer, stomach cancer, cancer of the bile ducts, cancer of the gall bladder, pancreatic cancer; cancer of the urinary tracts, including kidney, urothelium and bladder; cancers of the female genital tract, including cancer of the uterus, cervical cancer, ovarian cancer, choriocarcinoma and trophoblastoma; cancers of the male genital tract, including prostate cancer, cancer of the seminal vesicles, testicular cancer, germinal cell tumours; cancers of the endocrine glands, including thyroid cancer, pituitary cancer and cancer of the adrenal glands; skin cancers, including haemangiomas, melanomas, sarcomas, including Kaposi's sarcoma; brain tumours, nerve tumours, eye tumours, meningeal tumours, including astrocytomas, gliomas, glioblastomas, retinoblastomas, neurinomas, neuroblastomas, schwannomas, meningiomas; malignant haematopoietic tumours; leukaemias, (Acute Lymphocytic Leukaemia (ALL), Acute Myeloid Leukaemia (AML), Chronic Myeloid Leukaemia (CML), Chronic lymphocytic leukaemia (CLL)) chloromas, plasmocytomas, T or B cell leukaemias, Hodgkin or non-Hodgkin lymphomas, myelomas and various malignant haemopathies.

The compounds according to the invention may also be used for the preparation of medicaments, in particular for the preparation of a medicament for preventing or treating inflammatory diseases, such as, in particular, inflammatory diseases of the central nervous system, for instance multiple sclerosis, encephalitis, myelitis and encephalomyelitis, and other inflammatory diseases such as vascular pathologies, atherosclerosis, joint inflammations, arthrosis or rheumatoid arthritis.

The compounds according to the invention may therefore be used for the preparation of medicaments, in particular of medicaments for inhibiting casein kinase 1 epsilon and/or casein kinase 1 delta.

Thus, according to another of its aspects, a subject of the invention is medicaments which comprise a compound of formula (I), or an addition salt of the latter with a pharmaceutically acceptable acid, or alternatively a hydrate or a solvate of the compound of formula (I).

According to another of its aspects, the present invention relates to pharmaceutical compositions comprising, as active ingredient, a compound according to the invention. These pharmaceutical compositions contain an effective dose of at least one compound according to the invention or a pharmaceutically acceptable salt, a hydrate or a solvate of said compound, and also at least one pharmaceutically acceptable excipient.

Said excipients are chosen, according to the pharmaceutical form and the method of administration desired, from the usual excipients known to those skilled in the art.

In the pharmaceutical compositions of the present invention for oral, sublingual, subcutaneous, intramuscular, intravenous, topical, local, intratracheal, intranasal, transdermal or rectal administration, the active ingredient of formula (I) above, or the possible salt, solvate or hydrate thereof, may be administered in unit administration form, as a mixture with standard pharmaceutical excipients, to animals and to humans for the prophylaxis or treatment of the above disorders or diseases.

The suitable unit administration forms include oral administration forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, recta| administration forms, and implants. For topical application, the compounds according to the invention may be used in creams, gels, ointments or lotions.

By way of example, a unit administration form of a compound according to the invention in tablet form may comprise the following components:

| | | |
|---|---|---|
| Compound according to the invention | 50.0 | mg |
| Mannitol | 223.75 | mg |
| Sodium croscaramellose | 6.0 | mg |
| Maize starch | 15.0 | mg |
| Hydroxypropylmethylcellulose | 2.25 | mg |
| Magnesium stearate | 3.0 | mg |

When given orally, the dose of active ingredient administered per day may reach 0.1 to 20 mg/kg, in one or more dosage intakes.

There may be particular cases where higher or lower dosages are appropriate; such dosages do not depart from the context of the invention. According to the customary practice, the dosage appropriate to each patient is determined by the physician according to the method of administration and the weight and response of said patient.

According to another of its aspects, the present invention also relates to a method for treating the pathologies indicated above, which comprises the administration, to a patient, of an effective dose of a compound according to the invention or a pharmaceutically acceptable salt or hydrate or solvate thereof.

1. Compound of general formula (I) 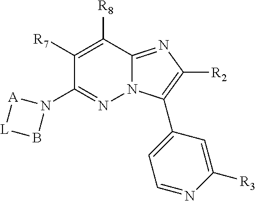
in which:
R₂ is a thienyl group or a furanyl group, optionally substituted with one or more substituents chosen from halogen atoms and C₁₋₆-alkyl groups;
R₃ is a hydrogen atom or a C₁₋₃-alkyl, —NR₄R₅, or C₁₋₄-alkyloxy group;
A is a C₁₋₇-alkylene group optionally substituted with one or two Rₐ groups;
B is a C₁₋₇-alkylene group optionally substituted with an R_{b} group;
L is either a nitrogen atom optionally substituted with an R_{c} or R_{d} group, or a carbon atom substituted with an Rₑ₁ group and an R_{d} group or two Rₑ₂ groups;
the carbon atoms of A and of B being optionally substituted with one or more R_{f} groups, which may be identical to or different from one another;
Rₐ, R_{b} and R_{c} are defined such that:
two Rₐ groups can together form a C₁₋₆-alkylene group;
Rₐ and R_{b} can together form a bond or a C₁₋₆-alkylene group;
Rₐ and R_{c} can together form a bond or a C₁₋₆-alkylene group;
R_{b} and R_{c} can together form a bond or a C₁₋₆-alkylene group;
R_{d} is a group chosen from a hydrogen atom and C₁₋₆-alkyl, C₃₋₇-cycloalkyl, C₃₋₇-cycloalkyl-C₁₋₆-alkyl, Cl₁₋₆-alkyloxy-C₁₋₆-alkyl, C₁₋₆-alkyloxy-C₁₋₆-alkyl, C₁₋₆-fluoroalkyl, benzyl and hydroxy-C₁₋₆-alkyl groups;
Rₑ₁ is an —NR₄R₅ group or a cyclic monoamine optionally comprising an oxygen atom, the cyclic monoamine being optionally substituted with one or more substituents chosen from a fluorine atom and C₁₋₆-alkyl, C₁₋₆-alkyloxy and hydroxyl groups;
Two Rₑ₂ form, with the carbon atom which bears them, a cyclic monoamine optionally comprising an oxygen atom, this cyclic monoamine being optionally substituted with one or more R_{f} groups, which may be identical to or different from one another;
R_{f} is a C₁₋₆-alkyl, C₃₋₇-cycloalkyl, C₃₋₇-cycloalkyl-C₁₋₆-alkyl, C₁₋₆-alkyloxy-C₁₋₆-alkyl, hydroxy-C₁₋₆-alkyl, C₁₋₆-fluoroalkyl or phenyl group;
R₄ and R₅ are, independently of one another, a hydrogen atom or a C₁₋₄-alkyl. C₃₋₇-cycloalkyl or C₃₋₇-cycloalkyl-C₁₋₆-alkyl group;
R₇ and R₈ are, independently of one another, a hydrogen atom or a C₁₋₆-alkyl group; in the form of a base or of an addition salt with an acid.

2. Compound of general formula (I), according to claim 1, characterized in that:
R₂ is a thienyl group, optionally substituted with one or more substituents chosen from halogen atoms and C₁₋₆-alkyl groups.

3. Compound of general formula (I), according to claim 1, characterized in that:
R₂ is a furanyl group, optionally substituted with one or more substituents, which may be identical to or different from one another, chosen from halogen atoms and C₁₋₆-alkyl groups.

4. Compound of general formula (I), according to any one of claims 1 to 3, characterized in that:
R₃ is a hydrogen atom or a group chosen from C₁₋₃-alkyl groups and —NR₄R₅ groups,
R₄ and R₅ are, independently of one another, a hydrogen atom or a C₁₋₄-alkyl group.

5. Compound of general formula (I), according to any one of claims 1 to 4, characterized in that:
R₇ and R₈ are a hydrogen atom.

6. Compound of general formula (I), according to any one of claims 1 to 5, characterized in that:
A is a C₁₋₇-alkylene group optionally substituted with one or two Rₐ groups;
B is a C₁₋₇-alkylene group optionally substituted with an R_{b} group;
L is either a nitrogen atom optionally substituted with an R_{c} or R_{d} group, or a carbon atom substituted with an Rₑ₁ group and an R_{d} group or two Rₑ₂ groups;
the carbon atoms of A and of B being optionally substituted with one or more R_{f} groups, which may be identical to or different from one another;
Rₐ. R_{b} and R_{c} are defined such that:
two Rₐ groups can together form a C₁₋₆-alkylene group;
Rₐ and R_{b} can together form a bond or a C₁₋₆-alkylene group;
Rₐ and R_{c} can together form a bond or a C₁₋₆-alkylene group;
R_{b} and R_{c} can together form a bond or a Cₘ-alkylene group;
R_{d} is a group chosen from a hydrogen atom and C₁₋₆-alkyl and hydroxy-C₁₋₆-alkyl groups;
Rₑ₁ is a cyclic monoamine;
two Rₑ₂ form, with the carbon atom which bears them, a monoamine, this cyclic monoamine being optionally substituted with one or more R_{f} groups, which may be identical to or different from one another;
R_{f} is a C₁₋₆-alkyl or hydroxy-C₁₋₆-alkyl group.

7. Compound of general formula (I), according to any one of claims 1 to 6, characterized in that:
the cyclic amine formed by —N-A-L-B— is a piperazinyl, hexahydropyrrolopyrrolyl, octahydropyrrolopyridinyl, diazaspiroundecyl or pyrrolidinylpiperidinyl group, optionally substituted with one or more groups chosen, independently of one another, from a C₁₋₆-alkyl group and a hydroxy-C₁₋₆-alkyl group.

8. Compound of general formula (I), according to any one of claims 1 to 7, characterized in that:
R₂ is a thien-2-yl, 5-methylthien-2-yl, 5-chlorothien-2-yl, thien-3-yl, 2,5-dimethylthien-3-yl, 2,5-dichlorothien-3-yl or furan-2-yl group;
R₃ is a hydrogen atom, a methyl group or an —NH₂ group;
R₇ and R₈ are a hydrogen atom;
the cyclic amine formed by —N-A-L-B— is a piperazin-1-yl, 3-methylpiperazin-1-yl, 4-methylpiperazin-1-yl, 3,3-dimethylpiperazin-1-yl, (cis)-3,5-dimethylpiperazin-1-yl, 4-(2-hydroxyethyl)piperazin-1-yl, 4-(2-hydroxy-2-methylpropyl)piperazin-1-yl, (cis)-hexahydropyrrolo[3,4-c]pyrrol-2(1H)-yl, (cis)-5-methylhexahydropyrrolo[3,4-c]pyrrol-2(1H)-yl, octahydro-6H-pyrrolo[3,4-b]pyridin-6-yl, 2,9-diazaspiro[5.5]undec-9-yl or 4-pyrrolidin-1-ylpiperidin-1-yl group;
in the form of a base or of an addition salt with an acid.

9. Process for preparing a compound of general formula (I) according to claim 1, characterized in that a compound of general formula (II) 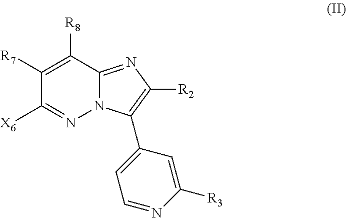
in which R₂, R₇ and R₈ are as defined according to claim 1 and X₆ is a halogen, is reacted with an amine of general formula (IIa) 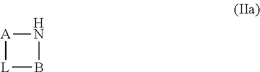
in which A, L and B are as defined according to claim 1.

10. Process for preparing a compound of general formula (I) according to claim 1, characterized in that a compound of general formula (V) 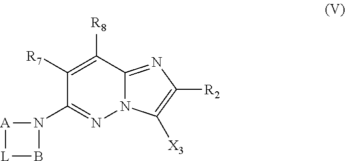
in which R₂, A, L, B, R₇ and R₈ are as defined according to claim 1 and X₃ is a halogen chosen from bromine and iodine, is reacted with a pyridine derivative of general formula (IIIa) 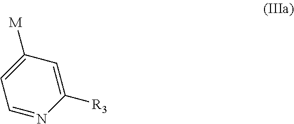
in which R₃ is as defined according to claim 1 and M is a group chosen from trialkylstannyl, dihydroxyboryl or dialkyloxyboryl groups.

11. Process for preparing a compound of general formula (I) according to claim 1, characterized in that a compound of general formula (VI) 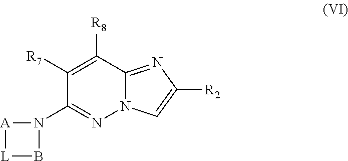
in which R₂, A, L, B, R₇ and R₈ are as defined according to claim 1, is reacted with a compound of general formula (VIa) 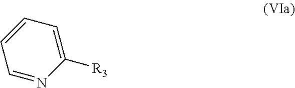
in which R₃ is a hydrogen atom or a C₁₋₃-alkyl group, so as to obtain a compound of general formula (V) 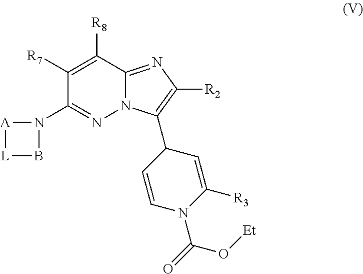
in which R₂, A, L, B, R₇ and R₈ are as defined according to claim 1, said compound of general formula (V) then being oxidized.

12. Medicament, characterized in that it comprises a compound of formula (I) according to any one of claims 1 to 8, in the form of a base or of an addition salt with a pharmaceutically acceptable acid.

13. Pharmaceutical composition, characterized in that it comprises a compound of formula (I) according to any one of claims 1 to 8, in the form of a base or of an addition salt with a pharmaceutically acceptable acid, and also at least one pharmaceutically acceptable excipient.

14. Use of a compound of general formula (I) according to any one of claims 1 to 8, for the preparation of a medicament for treating or preventing sleep disorders or circadian rhythm disorders.

15. Use of a compound of general formula (I) according to any one of claims 1 to 8, for the preparation of a medicament for treating or preventing bipolar disorders.

16. Use of a compound of general formula (I) according to any one of claims 1 to 8, for the preparation of a medicament for treating or preventing diseases associated with dependence on abuse substances.

17. Use of a compound of general formula (I) according to any one of claims 1 to 8, for the preparation of a medicament for treating or preventing diseases related to hyperphosphorylation of the tau protein.

18. Use of a compound of general formula (I) according to any one of claims 1 to 8, for the preparation of a medicament for treating or preventing diseases caused or exacerbated by cell proliferation.

19. Use of a compound of general formula (I) according to claim 17, characterized in that the cells are tumour cells.

20. Use of a compound of general formula (I) according to any one of claims 1 to 8, for the preparation of a medicament for treating or preventing inflammatory diseases.
